(19) United States
(12) Patent Application Publication
(10) Pub. No.: US 2007/0031908 A1
(43) Pub. Date: Feb. 8, 2007

(54) Method for detecting a progressive, chronic dementia disease, and corresponding peptides and detection reagents

(76) Inventors: Norbert Lamping, Hannover (DE); Hans Dieter Zucht, Hannover (DE); Hartmut Selle, Hannover (DE); Michael Jurgens, Hannover (DE); Gabriele Heine, Hannover (DE); Rudiger Hess, Hannover (DE); Markus Kellmann, Harpstedt (DE); Jens Lamerz, Hannover (DE); Thomas Mohring, Bremen (DE)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 10/549,141

(22) PCT Filed: Mar. 18, 2004
(86) PCT No.: PCT/EP04/02824

§ 371 Date: Jul. 3, 2006

(30) Foreign Application Priority Data
Mar. 18, 2003 (EP) 03005968.7
Jan. 7, 2004 (EP) 04000170.3

(51) Int. Cl. G01N 33/543

(52) U.S. Cl. 435/7.92; 436/518; 436/86

(57) ABSTRACT

The invention relates to a method for detecting progressive, chronic dementia diseases or a predisposition to such diseases or method for the prognosis of such diseases. For this purpose, the concentration of particular peptides in body fluids or other samples from the patient is measured in a method which can be carried out in a laboratory. The invention further relates to peptides which have been found for determining the presence and/or the grade of the progressive, chronic dementia disease. The invention additionally relates to detection reagents such as antibodies and nucleic acids and the like for detecting said peptides or the corresponding nucleic acids. The invention further relates to pharmaceutical products which comprise the peptides according to the present invention, antibodies directed to said peptides, nucleic acids corresponding to said peptides, peptide antagonists, or peptide agonists for the therapy, diagnosis, prognosis or prophylaxis of neurological diseases, in particular of Alzheimer's disease. The invention further relates to methods for stratifying patients or participants in clinical studies.

### FIELD OF THE INVENTION

The invention relates to a method for detecting progressive, chronic dementia diseases or a predisposition to such diseases or method for the prognosis of such diseases. For this purpose, the concentration of particular peptides in body fluids or other samples from the patient is measured in a method which can be carried out in a laboratory. The invention further relates to peptides which have been found for determining the presence and/or the grade of the progressive, chronic dementia disease.

The invention additionally relates to detection reagents such as antibodies and nucleic acids and the like for detecting said peptides or the corresponding nucleic acids. The invention further relates to pharmaceutical products which comprise the peptides according to the present invention, antibodies directed to said peptides, nucleic acids corresponding to said peptides, peptide antagonists, or peptide agonists for the therapy, diagnosis, prognosis or prophylaxis of neurological diseases, in particular of Alzheimer's disease. The invention further relates to methods for stratifying patients or participants in clinical studies.

### BACKGROUND OF THE INVENTION

Dementia diseases represent an increasing problem in industrialized countries because of the higher average life expectancy. Dementia diseases are in most cases incurable and make long-term and expensive care of the patients necessary. More than 60 dementia diseases are known, including diseases associated with manifestations of dementia. However, Alzheimer's disease (AD) accounts for about 65% of these, and the diagnosis and therapy thereof is therefore of great importance. Besides Alzheimer's disease, the following non-Alzheimer's dementias are known, inter alia: vascular dementia, Lewy body dementia, Binswanger dementia, and dementia diseases which occur as concomitant effects of other disorders such as Parkinson's disease, Huntington's disease, Pick's disease, Gerstmann-Sträussler-Scheinker disease, Creutzfeldt-Jakob disease, depression etc.

Alzheimer's disease is a neurodegenerative disease distinguished by the following symptoms: decline in intellectual abilities, confusion and diminished ability to look after oneself. A greatly restricted short-term memory in particular is characteristic of Alzheimer's disease. There are morphological changes in the brain manifested inter alia in the form of amyloid deposits and degenerated nerve cells. The morphological changes can be diagnosed histologically after the patient's death and are as yet the only reliable detection of the disease. These histopathological diagnoses are based on criteria fixed by the Consortium to Establish a Registry for Alzheimer's Disease (CERAD). The following criteria-based diagnostic systems are currently used to diagnose Alzheimer's disease: the International Classification of Diseases, 10th revision (ICD-10), the Diagnostic and Statistical Manual of Mental Disorders, 4th edition (DSM-IV) of the American Psychiatric Association, and the Work Group criteria drawn up by the National Institute of Neurological and Communicative Disorders Association NINCDS-ADRDA.

These systems use a number of neuropsychological tests in order to diagnose Alzheimer's disease, but not objectively measurable clinical parameters. It is of particular interest to establish the current level of the severity of the disease, which is possible for example through determination of the mini-mental score. The mini-mental score is determined with the aid of a mini-mental state examination (MMSE), a psychological test. This makes it possible inter alia to observe the course of the disease and the efficacy of any therapies. Clark et al. were able to show, however, that determination of the mini-mental score has only limited validity for determining the course of Alzheimer's disease, because large inaccuracies of measurement and wide variations in the level of the score occur [1]. The provision of a reliable, clinically measurable parameter able to supplement or replace the mini-mental state examination (MMSE) or other neuropsychological tests used to date for diagnosing diseases such as Alzheimer's disease is therefore of great medical, and thus also economic, importance. In addition, the provision of a clinical measurement parameter for determining preliminary stages of neurological diseases such as, for example, Mild Cognitive Impairment (MCI) is of great importance. Cognitive tests of these types are moreover carried out only by specialized centers. A biochemical test would have the advantage of being easily available for non-specialized physicians too.

At present, no causal therapy is available for the treatment of Alzheimer's disease. The disease is merely treated symptomatically, e.g. by administration of neurotransmitters such as acetylcholine or acetylcholinesterase inhibitors. Further possible therapeutic strategies being tested at present are the administration of antioxidants, of radical scavengers, of calcium channel blockers, of antiinflammatory substances, of secretase inhibitors, of anti-amyloid antibodies etc., and immunization against amyloid peptides. However, no causal therapy of this disease is yet possible.

WO 02/090974 has already disclosed a method of the generic type in which the presence and, where appropriate, the severity of a chronic dementia disease is indicated by a marker peptide. Other markers are also disclosed in WO 02/082075. However, there continues to be a great need for additional marker substances for this area of pathology which could be used inter alia for confirming the result in addition to or in combination with previously disclosed markers, which could possibly indicate other subtypes of the disease, which give additional information about the biochemical processes of the disease or which make a therapy possible through administration or blocking of particular peptides. Moreover, from the literature it becomes clear that a single marker is in most cases not sufficient for diagnosing a particular disease, but the use of at least two markers (a marker panel) which are correlated with each other allow for obtaining improved results in diagnosis. Therefore, a particular goal of the present invention is to provide for marker panels, comprising at least two markers, preferably derived from different proteins, to improve the diagnosis of chronic dementia diseases.

### SUMMARY OF THE INVENTION

The object of the invention is to provide further markers for diagnosing Alzheimer's disease to improve the diagnosis of chronic dementia diseases, to provide a method which can be used early and reliably for detecting chronic dementia diseases, in particular Alzheimer's disease, and to provide methods for the therapy of chronic dementia diseases.

It has surprisingly been found that the concentration of certain peptides, which are described in detail below, is changed in bodily fluid samples from patients suffering from Alzheimer's disease, especially in the cerebrospinal fluid, relative to their concentration in control samples, and thus detection of Alzheimer's disease is made possible. In particular, it has been found that a relationship exist between peptide(s)/protein(s) derived from different proteins. Some of these peptides identified according to the present invention which are suitable inter alia therefor are referred to hereinafter as Dementia Related Secretogranin 1 (DRES) peptides, thus, represent peptides derived from the protein secretogranin 1. Further peptides identified according to the present invention encompass peptides derived form chromogranin A, secretogranin 2 and/or secretogranin 5.

To achieve the object, the invention encompasses a method for detecting a neurological, in particular a chronic dementia disease, in particular Alzheimer's disease or a predisposition to such a disease through determination of one or more DRES peptides, SG1 peptides or SG1 proteins which are derived from the sequence having the GenBank Accesion No. NM_{—}001819 (SEQ ID 45) in an individual's liquid biological sample. Preferably, the above one or more peptides are determined in combination with the determination of one or more peptides derived from chromogranin A, secretogranin 2 and/or secretogranin 5. The results obtained by conducting this method can be used by a physician for diagnosis. Since it can be assumed that the detected substances are causally connected with the disease, the present invention also includes their administration or their blocking for the therapy of Alzheimer's disease or related neurological diseases.

To achieve the object according to the present invention, a method is provided for the detection or for the prognosis of a neurological disease, in particular a progressive, chronic dementia disease, in particular Alzheimer's disease, in which at least one SG1 protein, SG1 peptide or DRES peptide in a patient's liquid biological sample is determined. A further embodiment is the diagnosis of dementia diseases at an early time, e.g. in the diagnosis of mild cognitive impairment (MCI) or in neurological diseases different from Alzheimer's disease, such as, for example, Lewy body dementia, vascular dementia or depression. Preferably, the above method additionally comprises the determination of one or more peptides derived from chromogranin A, secretogranin 2 and/or secretogranin 5.

Various approaches to achieve this are possible and customary in medical diagnosis:

On the one hand it is possible generally to investigate for the presence of at least one marker peptide, and the absence or presence of this/these marker peptide(s) then allows the disease to be diagnosed.

In another diagnostic strategy, firstly the concentrations of the marker peptide(s) which are normally present in controls and in patients suffering from the disease to be diagnosed are determined and, on the basis of these measurements, a limiting value, often also called the cut-off point, which separates the group regarded as healthy from the group regarded as ill is determined. The limiting value determined individually for each marker peptide makes unambiguous differentiation of healthy and ill people possible.

In a further diagnostic strategy, a concentration increase or concentration reduction, which is specific for the particular marker peptide, of the marker peptide(s) in the patient's sample is determined relative to the concentration of the marker peptide(s) in the control sample, and significant marker peptide(s) concentration change is regarded as positive detection result for the disease. In this connection it is possible in principle for each defined individual peptide either to undergo only an increase in the concentration in the patient, or it is possible in principle for this peptide only to undergo a reduction in the concentration in the patient. For a defined individual peptide it is not possible for the peptide concentration simultaneously to be increased in one patient and to be reduced, relative to the control group, in another patient with the same disease.

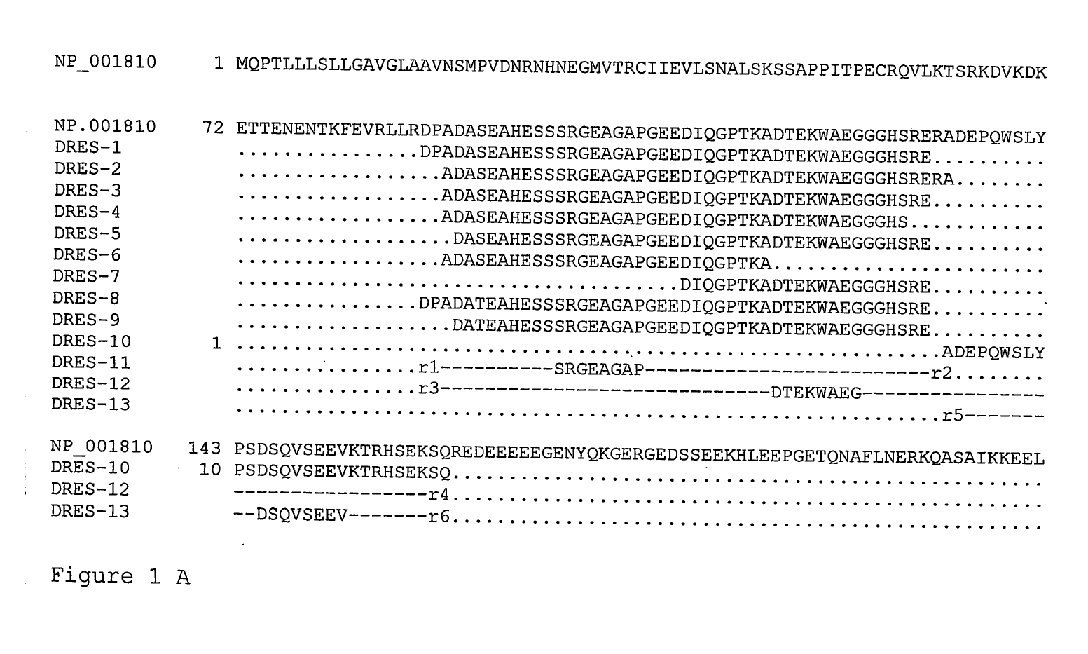

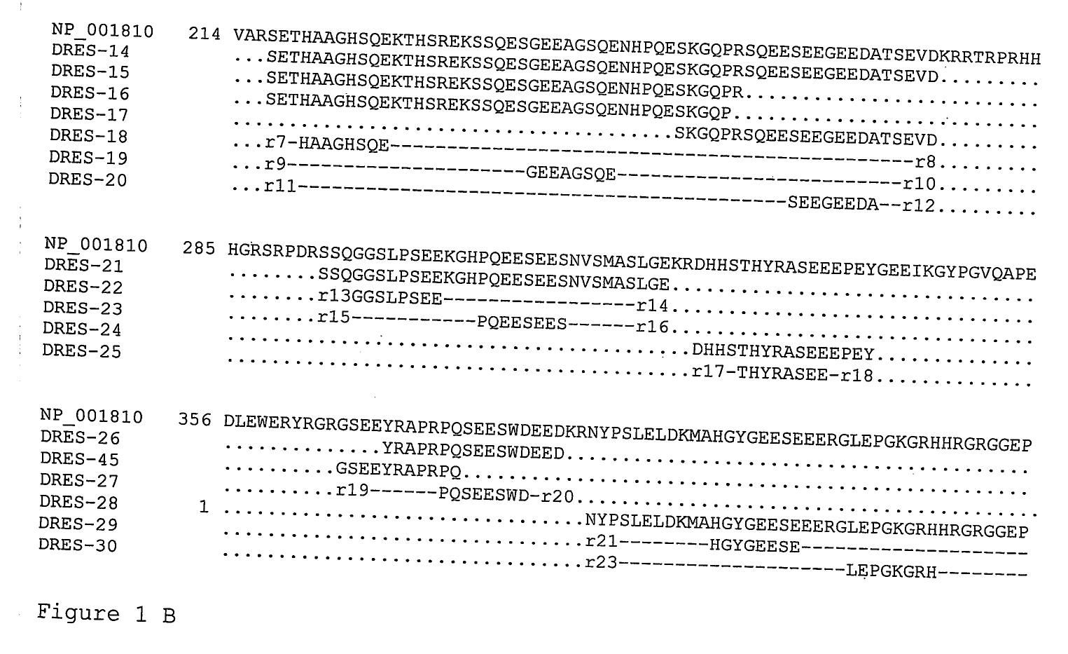

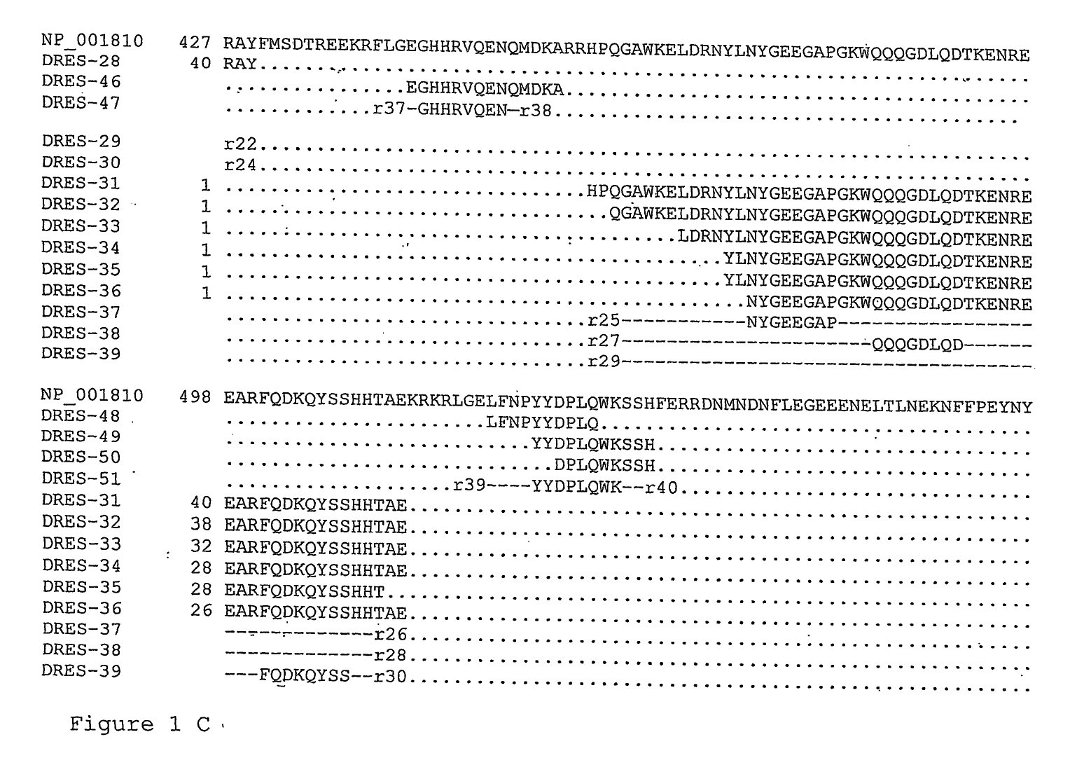

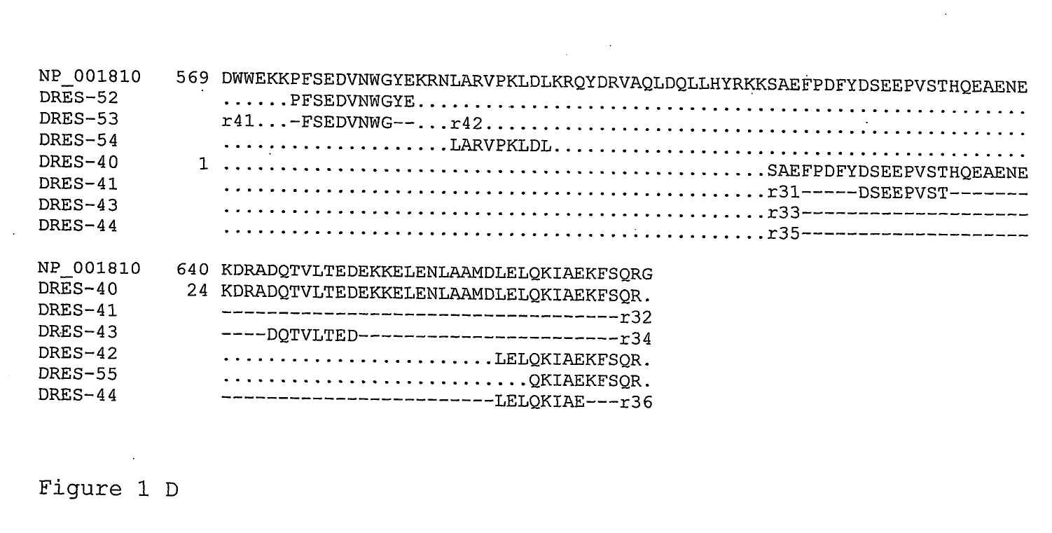

Preferred markers of the invention being derived from secretogranin 1 are indicated in the sequence listing and are called DRES-1 to DRES-55 corresponding to Seq. ID 1 to 44 and 47 to 57. The sequences of the DRES peptides are depicted in FIG. 1 and in Table 1. Assignment of the DRES peptides to their respective Seq. ID No. is shown in Table 1. Further preferred markers are derived from the proteins chromogranin A, secretogranin 2 and/or secretogranin 5, respectively. Said markers are depicted in table 1 and the sequence listing (Seq. ID 58 to 85).

Preferably, it is possible to correlate the concentration of the SG1 proteins, SG1 peptides or DRES peptides in the sample, or the characteristic pattern of the occurrence of a plurality of defined SG1 proteins, SG1 peptides or DRES peptides, with the severity, the prognosis or the probability of the occurrence of the disease. These novel markers therefore make it possible to develop and monitor therapies for the treatment of neurological diseases, in particular of chronic dementia diseases, in particular of Alzheimer's disease, because the course and any successful cure resulting from a therapy or a diminished progression of the disease can be established at an early time (surrogate marker). An effective therapy of Alzheimer's disease, one of the commonest neurological diseases, is not possible at present, which underlines the urgency of the provision of an early, sensitive detection method.

The detection according to the invention of at least SG1 proteins, SG1 peptides or DRES peptides additionally makes it possible to stratify patients and participants in clinical studies. This also makes it possible to develop and employ medically therapeutic agents and diagnostic aids which are effective only in sub groups of patients.

In a preferred embodiment, at least one of the peptides derived from chromogranin A, secretogranin 2 and/or secretogranin 5 as described herein are determined together with the above described detection of the SG1 proteins, SG1 peptides or DRES peptides, thus, allowing an improved diagnosis.

There are marked changes in the concentrations of these substances in patients with Alzheimer's disease compared with healthy people. A further aspect of the invention is therefore leveling the DRES concentrations or the concentrations of the peptides derived from chromogranin A, secretogranin 2 and/or secretogranin 5, respectively, in patients with Alzheimer's disease to normal concentrations. This method can be employed for the therapy of Alzheimer's disease or related neurological diseases. When the protein(s) or peptide(s) concentrations or of the described protein(s) or peptide(s) are increased, the concentration(s) of this/these substance(s) can be reduced by therapeutic administration of, for example, antibodies against these substances or specific antisense nucleic acids, ribozymes, RNAi (RNA-mediated interference) nucleic acid molecules or triplex nucleic acids or corresponding antagonists directed against these substances. These agents (antibodies, antisense nucleic acids etc.) can be produced by the skilled person on the basis of the amino acid and nucleic acid sequences of the described peptides or proteins with the aid of conventional techniques [2, 3]. Substances which suppress the endogenous expression of SG1, chromogranin A, secretogranin 2 and/or secretogranin 5 proteins or their corresponding processed peptides, e.g. to SG1 peptides or DRES peptides can also be administered for the therapy. If the disease is caused by a deficiency of any one of the described proteins or peptides, e.g. SG1 protein, SG1 peptide or DRES peptide, therapeutic doses of said protein(s) or peptide(s) or corresponding agonists can be given. Substances which influence the processing of said proteins can also be employed therapeutically. As can be seen in FIG. 1, for example DRES-21 and DRES-24, and DRES-26 and DRES-28, are separated from one another by two basic amino acids (lysine and arginine). Such so-called “dibasic sequences” are frequently the points of attack of proteases which are involved in the processing of proteins to biologically active peptides. Many other DRES peptides are flanked at the N and/or C terminus by such di- or tribasic sequences, as is likewise evident from FIG. 1. Examples thereof are: DRES-14, DRES-17, DRES-31, DRES-32, DRES-33, DRES-34, DRES-36 and DRES-40. The same is true for the other proteins, namely chromogranin A, secretogranin 2 and secretogranin 5. Said proteins also contain these dibasic sequences desribed above, and, consequently, peptides derived from said proteins by natural processing are within the scope of the present invention.

Combination of different therapeutic strategies is, of course, also possible and appropiate in some circumstances.

The invention therefore also encompasses the use of SG1 proteins, SG1 peptides, DRES peptides, and corresponding peptidomimetics for the treatment of neurological diseases, especially Alzheimer's disease. It is also possible to use corresponding agonists, antagonists, and antibodies directed against SG1 proteins, SG1 peptides or DRES peptides for the direct or indirect modulation of the concentration of SG1 proteins, SG1 peptides and DRES peptides. Alternative to antibodies, it is also possible to use antibody fragments, antibody fusion proteins or other substances which bind selectively to SG1 proteins, SG1 peptides or DRES peptides. It is also possible as an alternative to said proteins and peptides to use fusion proteins of these proteins and peptides. Two or more proteins/peptides or one or more proteins/peptides can also be fused with further non-SG1 peptides. Examples of possible fusion partners are, for example, the HIV Tat or His tag sequences. These peptides may be linked either covalently or non-covalently, and it is possible to produce both linear and is branched or circular molecules from these peptides. One example of a non-covalent linkage would be, for example, the linkage of a biotin-labeled DRES peptide to a streptavidin-labeled antibody. The invention further encompasses also the use of antisense nucleic acids, triplex nucleic acids, RNAi nucleic acid molecules, ribozymes and other nucleic acids which modulate the expression of said proteins and peptides. The invention additionally encompasses agonists and antagonists which modulate the activity of said proteins and peptides.

The above considerations apply mutadis mutandis to chromogranin A, secretogranin 2 and secretogranin 5 and the corresponding peptides derived therefrom.

A further embodiment of this invention is the pharmaceutical formulation or chemical modification of the described proteins, peptides and nucleic acids to make it possible for them to cross the blood-brain barrier and/or the blood-CSF barrier more efficiently. They are thus made particularly suitable for therapeutic use. In order to achieve this, it is possible for example for the described peptides, proteins, nucleic acids, agonists or antagonists to be modified so that, for example, they become more lipophilic, favoring entry into the subarachnoid space. This can be achieved by introducing hydrophobic molecular constituents such as, for example, hydrocarbon chains such as ethylene glycol polymers (=pegylation) or else by “packaging” the substances in hydrophobic agents, e.g. liposomes. It is additionally possible to attach for example foreign peptide sequences which favor entry of the peptides, proteins, nucleic acids, agonists or antagonists according to the present invention into the subarachnoid space, or conversely impede emergence from the subarachnoid space, or which facilitate for example penetration into the interior of the cell, which can be brought about for example by the HIV Tat sequence.

The invention also encompasses the administration of said therapeutic agents by various routes such as, for example, as intravenous injection, as substance which can be administered orally, as inhalable gas or aerosol, as topical application or administration in the form of direct injections into the subarachnoid space, or into tissues such as muscle, fat, brain etc. It is possible in this way to achieve increased bloavailability and efficacy, and an increased local concentration of these therapeutic agents. For example, peptides or proteins which are administered orally can be protected by acid-resistant capsules from proteolytic degradation in the stomach. Very hydrophobic substances can become more hydrophilic and thus better suited for, for example, intravenous injections by suitable pharmaceutical processing, e.g. by pegylation, etc. Further possible dosage forms are inter alia the packaging of the active ingredients in polymers or gels (Atrix Labs, Fort Collins, Colo., USA, Andrx Pharmaceuticals, Davie, Fla. USA) etc.

A further embodiment of the invention is the use of the peptides and proteins according to the present invention, e.g. the use of the DRES peptides, SG1 peptides or SG1 proteins in screening methods in order to identify diagnostic aids or therapeutic agents for neurological diseases. It is possible by such screening methods to find, for example, molecules which activate or inhibit SG1 proteins, SG1 peptides or DRES peptides, or receptors of these substances can be found. Receptors identified in this way can be modulated by administering agonists or antagonists, which is expedient for the therapy of neurological diseases, especially of Alzheimer's disease.

In another preferred embodiment a panel of marker peptides is used in the diagnosis of neurological diseases, in particular in the diagnosis of Alzheimer's disease. Thus, a marker panel is within the scope of the present invention comprising a combination of at least two peptides derived from different proteins selected from the group of chromogranin A, secretogranin 1, secretogranin 2 and secretogranin 5. Preferably, at least one peptide of each protein is determined. More preferably, the marker panel comprises a peptide selected from the group of DRES peptides 1 to 44 and ID 47 to 57, of Seq. ID 58 to 60 derived from secretogranin 5, of Seq. ID 61 to 71 derived from secretogranin 2, and of Seq. ID 72 to 85 derived from chromogranin A. Most preferably, the marker panel comprises the Seq. ID 1, 58, 61 and 85.

Definitions:

Alternative Names of Secretogranin 1:

Various names are used in the literature for the protein secretogranin 1. Inter alia, secretogranin 1 is known under the following names and abbreviations: chromogranin B, CGB, CgB, CHGB, secretogranin 1, Sgl, SCG1, SG1. In addition, various peptides derived from secretogranin 1 are known. Some of them are in the following list: secretolytin, GWAK peptide, PE-11, CCB, BAM-1745. It is possible that further names, which are not listed here, are present in the literature for secretogranin 1.

Secretogranin 1, SG1 Protein

SG1, SG1 protein or secretogranin 1 is written in the present application when merely the complete amino acid sequence corresponding to NM_{—}001819 is meant. Also included in this definition are SG1 protein variants which are at least 70%, in particular 80%, in particular 90%, in particular 95%, homologous to the amino acid sequence corresponding to NM_{—}001819. These SG1 protein variants may on the one hand be of natural origin, i.e. translation products of SG1 protein mutants, SG1 protein alleles (same gene locus), SG1 protein homologs (different gene loci) or SG1 protein orthologs (different organisms) which occur thus in nature. On the other hand, these SG1 protein variants may also have been produced in an unnatural way, e.g. by site-directed mutagenesis techniques or by random mutagenesis, which can be induced for example by chemicals such as dimethyl sulfate or by ionizing radiation. The corresponding nucleic acid variants can be produced by substitutions, deletions, insertions or inversions. They may relate both to coding regions of the nucleic acid sequence and to noncoding regions such as, for example, promoters, introns, 3′- or 5′-untranslated RNA regions etc. The variations may be conservative, i.e. not altering the amino acid sequence, and non-conservative, i.e. lead to alteration of the amino acid sequence. The resulting SG1 protein variants may be both functional SG1 proteins and SG1 proteins which have restricted function or are inactive. Particularly included are also SG1 protein variants which occur on the basis of neurological diseases, in particular chronic dementia diseases, in particular Alzheimer's disease. Both SG1 proteins with and without signal sequence, pro forms of SG1 proteins which have not yet been processed, and already processed SG1 proteins, soluble SG1 proteins and membrane-associated SG1 proteins are included. Also included are variations of the SG1 protein sequence which are produced by alternative splicing, by alternative translation starting and termination points, by RNA editing, by alternative post-translational modifications, by translation of stop codons into unusual amino acids such as, for example, seleno-cysteine or pyrrolysine, and by further naturally occurring mechanisms.

SG1 Peptides, Secretogranin 1 Peptides

By definition, SG1 peptides or secretogranin 1 peptides are all substances which are fragments of secretogranin 1 or SG1 proteins. It is likewise true of SG1 peptides that they are at least 70%, in particular 80%, in particular 90%, in particular 95%, homologous to the amino acid sequence corresponding to NM_{—}001819. The homology is moreover calculated in accordance with the description in the paragraph “Homology of sequences” hereinafter. The % value for the homology is based on the respective SG1 peptides, i.e. an SG1 peptide with a length of 100 amino acids must be homologous in at least 70 of its amino acids to the sequence corresponding to NM_{—}001819. SG1 peptide variants are also included, corresponding to the SG1 protein variants, in this definition of SG1 peptides.

DRES Peptides:

Specific SG1 peptides are referred to hereinafter as DRES (“dementia-related secretogranin 1”) peptides if they can be detected in biological samples, see Seq. ID 1 to 44 and ID 47 to 57. DRES peptides are not arbitrary fragments of the complete secretogranin 1 but are peptides produced in a natural way. The term “produced in a natural way” means for the purposes of this application that DRES peptides are produced without adding proteases to the samples. This means that DRES peptides are either already produced in vivo or they are produced during sampling and analyzing the samples but without adding proteases to the sample. DRES peptides are derived from the SG1 sequence NM_{—}001819 mentioned at the outset. Alternatively, DRES peptides may also be derived from other database entries for SG1, such as, for example, BC000375 or Y00064 or further SG1 entries which are already known at present or which will be known in future. It is possible in this connection for the SG1 nucleic acid sequences and the protein sequences derived therefrom that they may differ from the sequence of the “GenBank” entry with the number NM_{—}001819. Two DRES peptides, DRES-8 and DRES-9, which are not derived from the sequence corresponding to the database entry NM_{—}001819 but from another SG1 sequence are claimed in this application. SG1 sequence entries may also be present in other sequence databases different from “GenBank”. Consequently, DRES peptides, SG1 peptides and SG1 proteins need not coincide exactly with the sequence corresponding to the entry in the “GenBank” sequence database with the accession no. NM_{—}001819. In addition, DRES peptides may comprise two point-mutated, two deleted or two additionally internally inserted amino acids, and N-terminal and/or C-terminal extensions. However, in these cases they must retain at least 8 amino acids from the secretogranin 1 sequence. The only amino acids suitable as N— or C-terminal extensions are those occurring in the secretogranin 1 sequence at this sequence position. Methods used for determining whether at least 8 amino acids of the DRES peptide coincide with the secretogranin 1 sequence are described in the following paragraph “Homology of sequences”.

The above considerations apply mutadis mutandis to chromogranin A, Acc. No A28468 (SEQ ID 88), secretogranin 2, Acc. No. NP_{—}003460 (SEQ ID 87), and secretogranin 5, Acc. No. NP_{—}003011 (SEQ ID 86), and corresponding peptides derived therefrom.

Homology of Sequences

The homology between sequences can be determined by using computer programs such as, for example, the GCG program package (Genetics Computer Group, University of Wisconsin, Madison, Wis., USA), including GAP [4], BLASTP, BLASTN, FASTA [5] or the well-known Smith-Waterman algorithm for determining homologies. Preferred parameters for the amino acid sequence comparison comprise the algorithm of Needleman and Wunsch [6], the comparison matrix BLOSUM 62 [7], a gap penalty of 12, a gap length penalty of 4 and a homology threshold (threshold of similarity) of 0. The GAP program is also suitable for use with the aforementioned parameters. The aforementioned parameters are the default parameters for amino acid sequence comparisons, where gaps at the ends do not reduce the homology level. With sequences which are very short compared with the reference sequence, it may additionally be necessary to increase the expectation value as far as 100 000 and, where appropriate, to reduce the word size as far as 2. Further exemplary algorithms, gap opening penalties, gap extension penalties, comparison matrices including those mentioned in the program handbook, Wisconsin package, version 9, September 1997, can be used. The selection will depend on the comparison to be carried out and also on whether the comparison is carried out between sequence pairs, in which case GAP or Best Fit are preferred, or between a sequence and a comprehensive sequence database, in which case FASTA or BLAST are preferred. An agreement of 70% found with the abovementioned algorithm is referred to as 70% homology for the purposes of this application. Corresponding statements apply to higher or lower degrees of homology.

Chemically or Post-Translationally Modified Peptides

A chemically or post-translationally modified peptide according to the present invention may consist both of D- and of L-amino acids, and of combinations of D- and L-amino acids, and may either occur naturally, be produced recombinantly or enzymatically or be synthesized chemically. These peptides may additionally comprise unusual amino acids, i.e. amino acids which do not belong to the 20 standard amino acids. Numerous examples of unusual amino acids and post-translational modifications such as, for example, phosphorus and sulfate groups, glycosylations, amidations, deamidations, pyroglutamate modifications etc. are described in the literature and databases [8]. For example, SG1 or DRES peptides have been found with modifications such as, for example, phosphorylations/sulfations and pyroglutamate modifications, and oxidized or amidated peptides have been determined. DRES-14 has been found with no or with one, two or three phosphate groups/sulfate groups, DRES-15, DRES-16 and DRES-36 have been found with and without a phosphate group/sulfate group, DRES-21 has been found as peptide oxide with a phosphate group and as peptide oxide without a phosphate group and DRES-32 has been found with an N-terminal pyroglutamate modification and DRES-42 as C-terminal amide.

Nucleic Acids

The nucleic acids corresponding to the above mentioned proteins and peptides according to the present invention are regarded as being DNA, RNA and DNA-RNA hybrid molecules both of natural origin, and nucleic acids which are produced synthetically, enzymatically or recombinantly and code for the respective proteins and peptides. Said nucleic acids may also be a constituent of vectors, especially of plasmids, cosmids, phage particles, artificial chromosomes, viral vectors, retroviruses, adenoviruses, adeno-like viruses or baculoviruses. The nucleic acids and vectors may also have a linear or circular structure. Also included are nucleic acids and vectors which are composed wholly or partly of modified nucleotides in which, for example, modifications are present in the base portion, in the sugar portion or in the phosphate portion. Such modifications, which often have a stabilizing effect, are already used inter alia in ribozyme, antisense, RNAi and triplex nucleic acid techniques.

Peptidomimetics:

Peptidomimetics or peptide mimetics are molecules which have the activity of the corresponding peptide or protein but are not build from the standard set of 20 amino acids but from other structures such as for example beta-amino acids, D-amino acids, unusual acids, other structures such as spiegelmers® (NOXXON Pharma AG, Berlin, Germany) or other non-amino acid structures which can substitute amino acid structures. Also the peptide backbone may be modified by substitution of the peptide bond by other chemical structures, for example by using sulfur or phosphorus instead of nitrogen within the peptide bond or by replacing certain carbon atoms by nitrogen resulting in for example azapeptides or by altering the flexibility of the peptide structure for example by introducing covalent bonds between amino acid side chains, etc. Also the terminal ends of the peptidomimetic can be altered for example by boronic acid at the C-terminal end of a peptide. Peptidomimetics may contain normal peptide structures in combination with peptidomimetic structures. Preferably those peptidomimetics are choosen, which have a good metabolic stability, a good bioavailability, and which closely resemble the activity or function of the corresponding natural peptide and have minimal side effects such as toxicity.

Significance:

The term significant is used in the sense in which the term significance is used in statistics. In this patent application, an error probability of less than 10%, preferably 5%, further preferably 1%, is defined as significant.

Sensitivity:

Sensitivity is defined as the proportion of patients with the disease who acquire a positive diagnostic result in a diagnosis for the disease, i.e. the diagnosis correctly indicates the disease.

Specificity:

Specificity is defined as the proportion of healthy people who acquire a negative diagnostic result in a diagnosis for the disease, i.e. the diagnosis correctly indicates that no disease is present.

Granin Family—Biological Background

The granin family of proteins (secretogranini chromogranin) is a group of acidic, secretory proteins which are present in the secretory granules of various endocrine and neuronal cells [9]. The granin family of proteins includes chromogranin A, secretogranin 1 (chromogranin B), secretogranin 2 (chromogranin C), secretogranin 3 (1B075), secretogranin 4 (HISL-19), secretogranin 5 (7B2) and secretogranin 6 (NESP55). Many granins, including secretogranin 1, have numerous dibasic or multibasic sequences, have a high proportion of acidic amino acid side chains (glutamic acid and aspartic acid), are thermally stable and bind calcium [9]. They have several calcium binding sites of high capacity but low affinity. These domains have homologies with the calcium-binding domains of, for example, calmodulin. Besides calcium, it is also possible for catecholamines, adenosine triphosphate (ATP) and other low molecular weight substances to interact with granins. These interactions possibly induce the aggregation of granins, which in turn coaggregate with peptide hormones and neuropeptides.

Secretogranin 1 is frequently also referred to as chromogranin B. It has a lo molecular weight of 76 kDa and has N- and O-glycosylations, and sulfate and phosphate groups, as post-translational modification. Chromogranin A is also known as CGA, secretory protein I, parathyroid secretory protein or PSP. The biological function of chromogranin A is not yet clear but it might be involved in catecholamine storage and release. There are known fragments of chromogranin A with certain activities. One of these chromogranin A fragments is pancreastatin, which may be important for the physiologic homeostasis of blood insulin levels. Another known chromogranin A fragment is chromostatin. Chromogranin A is a phosphorylated, glycosylated protein with an amidated C-terminus and ist concentration is frequently increased in patients suffering from various types of cancer.

Alternative name of secretogranin 2 is chromogranin C. The molecular weight of secretogranin 2 is about 86 kDa. It is suggested that secretogranin is involved in the packaging or sorting of peptide hormones and neuropeptides into secretory vesicles. Secretoneurin is a fragment of secretogranin 2 which exerts chemotactic effects on certain cell types.

Secretogranin 5 is also known as secretory granule neurodoctrine protein 1, SGNE 1, pituitary polypeptide 7B2,P7B2 or 7B2 protein. Secretogranin 5 has a molecular weight of 21 kDa. Secretogranin functions as a chaperone specific for proprotein convertase-2 (PC2). The C-terminus of secretogranin 5 can inhibit PC2 activity. In addition, secretogranin 5 has functions in the regulation of pituitary hormone secretion.

The numerous dibasic sequences present in the granins might possibly represent competitive substrates for proteases, e.g. prohormone convertases (PC1, PC2, PC3, furin), which cause the processing of peptide hormones and neuropeptides. Secretogranin 5 (7B2) additionally binds directly to the pro form of PC2 and a C-terminal peptide of secretogranin 5 inhibits PC2. Granins might thus possibly modulate the processing of peptide hormones and neuropeptides. It has additionally been reported that processing of chromogranin A results in various biologically active peptides such as vasostatin I, catestatin, pancreastatin, betagranin and chromostatin. These peptides have various functions such as, for example, regulation of carbohydrate metabolism, vasodilating and bacteriolytic functions, and promotion of the survival of sensory neurons.

To date only chromogranin A of the granins has been used for diagnosis. Detection of elevated chromogranin A concentrations is used to diagnose various endocrine and neuroendocrine tumors, in which cases chromogranin A concentrations increased by up to 1000-fold are found [10]. In addition chromogranin A concentrations which are too high occur in a particular, genetically related variant of high blood pressure [11], in patients with end-stage renal failure and in patients suffering from heart failure.

Biological functions of peptides derived from secretogranin 1 are known only in the case of secretolytin, a defined proteolytic fragment of secretogranin 1. Secretolytin displays bacteriolytic activity [13].

### Preferred Embodiments of the Invention

The dementia detected by the method of the invention is preferably a progressive, chronic dementia disease such as, for example, Alzheimer's disease. It has been possible to date to detect the change in the concentration of DRES peptides according to the invention in patients suffering from Alzheimer's disease. Moreover, it is considered that detecting not only one or more DRES peptides but also at least one additional marker derived from chromogranin A, secretogranin 2 and/or secretogranin 5 as shown herein results in superior diagnostic methods for chronic dementia like Alzheimer's disease. The use of marker panels, like marker panels exemplified herein, allows for improvements in diagnosis and therapy of the mentioned diseases. It can be concluded from this that the peptides of the invention can also be used for the detection and for the therapy of Alzheimer's disease and related neurological diseases. One embodiment of this method is determination of dementia diseases at an early time, such as, for example, detection of mild cognitive impairment (MCI).

The determination is preferably concentrated on particular fragments of secretogranin 1 having the GenBank Accesion No. NM_{—}001819, i.e. on peptides which represent partial sequences of secretogranin 1 or else on complete SG1. These specific peptides are referred to as dementia related secretogranin 1 (DRES) peptides and are referred to hereinafter as DRES-1 to DRES-55. The connection between SG1 protein and DRES-1 to DRES-55 is depicted in FIG. 1. The sequences we found for the peptides are indicated in the sequence listing. The DRES peptides determined by us are produced naturally in nature and have not to date been described in the literature, or at least not in connection with neurological diseases. These DRES peptides are different from SG1 fragments derived from in vitro proteolysis after addition of proteases such as, for example, trypsin as described in the literature. Therefore they represent novel, previously unknown substances which are produced naturally in nature. DRES peptides are not substances produced by human manipulation of secretogranin 1. The DRES peptides were initially concentrated and purified from biological samples by reverse phase chromatography and subsequently separated by mass spectrometry from other accompanying proteins, so that it was subsequently possible to sequence them.

The sequences of the DRES peptides in the single-letter amino acid code are shown in table 1:

Furthermore, the present invention relates to marker panels and their use in diagnosis of chronic dementia diseases, e.g. Alzheimer's disease. The marker panel comprises at least one marker derived from secretogranin 1, e.g. a marker peptide selected from the DRES peptides as disclosed herein, in concert with at least one additional marker derived from chromogranin A, secretogranin 2 and/or secretogranin 5, e.g. a marker peptide as disclosed in table 1 below.

| | | |
|---|---|---|
| Amino | Mass | Modifi- |

| | | | |
|---|---|---|---|
| Seq. | acid | (Da) | cation |

| | | | | |
|---|---|---|---|---|
| ID | position | *, ** | *** | Sequence |

Secretogranin 1

| | | | | |
|---|---|---|---|---|
| 1 | 88-132 | 4605.0 | — | DPADASEAHSSSSRGEAGA |
| | | | | PGEEDIQGPTKADTEKWAE |
| | | | | GGGHSRE |
| 2 | 90-134 | 4620.1 | — | ADASEAHESSSRGEAGAPG |
| | | | | EEDIQGPTKADTEKWAEGG |
| | | | | GHSRERA |
| 3 | 90-132 | 4392.9 | — | ADASEAHESSSRGEAGAPG |
| | | | | EEDIQGPTKADTEKWAEGG |
| | | | | GHSRE |
| 4 | 90-130 | 4107.8 | — | ADASEAHESSSRGEAGAPG |
| | | | | EEDIQGPTKADTEKWAEGG |
| | | | | GHS |
| 5 | 91-132 | 4321.9 | — | DASEAHESSSRGEAGAPGE |
| | | | | EDIQGPTKADTEKWAEGGG |
| | | | | HSRE |
| 6 | 90-118 | 2853.3 | — | ADASEAHESSSRGEAGAPG |
| | | | | EEDIQGPTKA |
| 7 | 111-132 | 2368.1 | — | DIQGPTKADTEKWAEGGGH |
| | | | | SRE |
| 8 | 88-132 | 4619.0 | S93 to | DPADATEAHESSSRGEAGA |
| | | | T93 | PGEEDIQGPTKADTEKWAE |
| | | | | GGGHSRE |
| 9 | 91-132 | 4335.9 | S93 to | DATEAHESSSRGEAGAPGE |
| | | | T93 | EDIQGPTKADTEKWAEGGG |
| | | | | HSRE |
| 10 | 134-161 | 3246.5 | — | ADEPQWSLYPSDSQVSEEV |
| | | | | KTRHSEKSQ |
| 11 | 102-109 | ** ≧686.3 | — | r1-SRGEAGAP-r2 |
| 12 | 119-126 | ≧934.4 | — | r3-DTEKWAEG-r4 |
| 13 | 145-152 | ≧891.4 | — | r5-DSQVSEEV-r6 |
| 14 | 217-275 | 6433.7 | *** with | SETHAAGHSQEKTHSREKS |
| | | | 0, 1, 2, | SQESGEEAGSQENHPQESK |
| | | | or 3 | GQPRSQEESEEGEEDATSE |
| | | | phos/ | VD |
| | | | sul | |
| 15 | 217-257 | 4583.1 | with 0 | SETHAAGHSQEKTHSREKS |
| | | | or 1 | SQESGEEAGSQENHPQESK |
| | | | phos/ | GQPR |
| | | | sul | |
| 16 | 217-257 | 4427.0 | with 0 | SETHAAGHSQEKTHSREKS |
| | | | or 1 | SQESGEEAGSQENHPQESK |
| | | | phos/ | GQP |
| | | | sul | |
| 17 | 253-275 | 2522.1 | — | SKGQPRSQEESEEGEEDAT |
| | | | | SEVD |
| 18 | 220-227 | ≧835.4 | — | r7-HAAGHSQE-r8 |
| 19 | 240-247 | ≧805.3 | — | r9-GEEAGSQE-r10 |
| 20 | 263-270 | ≧864.3 | — | r11-SEEGEEDA-r12 |
| 21 | 293-323 | 3202.4 | M₃₁₈ = | SSQGGSLPSEEKGHPGEES |
| | | without | ox. | EESNVSMASLGS |
| | | mod., | M₃₁₈ = | |
| | | ox: | ox. | |
| | | 3218.4 | S₃₁₁ = | |
| | | ox. + | phos. | |
| | | phos. | | |
| | | 3298.4 | | |
| 22 | 296-303 | ≧774.3 | — | r13-GGSLPSEE-r14 |
| 23 | 307-314 | ≧933.4 | — | r15-PQEESEES-r16 |
| 24 | 326-341 | 1985.8 | — | DHHSTHYRASEEEPEY |
| 25 | 330-337 | ≧991.4 | — | r17-THYRASEE-r18 |
| 26 | 370-385 | 1992.8 | — | YRAPRPQSEESWDEED |
| 27 | 375-382 | ≧976.4 | — | r19-PQSEESWD-r20 |
| 28 | 388-429 | 4750.2 | — | NYPSLELDKMAHGYGEESE |
| | | | | EERGLEPGKGRHHRGRGGE |
| | | | | PRAY |
| 29 | 399-406 | ≧906.3 | — | r21-HGYGEESE-r22 |
| 30 | 411-418 | ≧892.5 | — | r23-LEPGKGRH-r24 |
| 31 | 459-513 | 6499.0 | — | HPQGAWKELDRNYLNYGEE |
| | | | | GAPGKWQQQGDLQDTKENR |
| | | | | EEARFQDKQYSSHHTAE |
| 32 | 461-513 | 6264.9 | Q₄₆₁ | QGAWKELDRNYLNYGEEGA |
| | | | pyro- | PGKWQQQGDLQDTKENREE |
| | | | Glu | ARFQDKQYSSHHTAE |
| 33 | 467-513 | 5565.6 | — | LDRNYLNYGEEGAPGKWQQ |
| | | | | QGDLQDTKENREEARFQDK |
| | | | | QYSSHHTAE |
| 34 | 471-513 | 5067.3 | — | YLNYGEEGAPGKWQQQGDL |
| | | | | QDTKENREEARFQDKQYSS |
| | | | | HHTAE |
| 35 | 471-511 | 4867.2 | — | YLNYGEEGAPGKWQQQGDL |
| | | | | QDTKENREEARFQDKQYSS |
| | | | | HHT |
| 36 | 473-513 | 4791.2 | with and | NYGEEGAPGKWQQQGDLQD |
| | | | without | TKENREEARFQDKQYSSHH |
| | | | phos/ | TAE |
| | | | sul | |
| 37 | 473-480 | ≧835.3 | — | r25-NYGEEGAP-r26 |
| 38 | 484-491 | ≧930.4 | — | r27-QQQGDLQD-r28 |
| 39 | 502-509 | ≧1001.4 | — | r29-FQDKQYSS-r30 |
| 40 | 617-676 | 6970.3 | — | SAEFPDFYDSEEPVSTHQE |
| | | | | AENEKDRADQTVLTEDEKK |
| | | | | ELENLAAMDLELQKIAEKF |
| | | | | SQR |
| 41 | 625-632 | ≧862.4 | — | r31-DSEEPVST-r32 |
| 42 | 664-676 | 1588.9 | C-term. | LELQKIAEKFSQR |
| | | | amidated | |
| | | | or as | |
| | | | acid | |
| 43 | 644-651 | ≧919.4 | — | r33-DQTVLTED-r34 |
| 44 | 664-671 | ≧942.5 | — | r35-LELQKIAE-r36 |
| 47 | 366-376 | 1288.6 | | GSEEYRAPRPQ |
| 48 | 443-456 | 1677.8 | | EGHHRVQENQDMDKA |
| 50 | 521-530 | 1268.6 | | LFNPYYDPLQ |
| 51 | 525-535 | 1422.7 | | YYDPLQWKSSH |
| 52 | 527-535 | 1096.5 | | DPLQWKSSH |
| 54 | 575-585 | 1341.5 | | PFSEDVNWGYE |
| 56 | 589-597 | 1023.6 | | LARVPKLDL |
| 57 | 667-676 | 1233.7 | | QKIAEKFSQR |
| 49 | 444-451 | ≧975.5 | | r37-GHHRVQEN-r38 |
| 53 | 525-532 | ≧1111.5 | | r39-YYDPLQWK-r40 |
| 55 | 576-583 | ≧952.4 | | r41-FSEDVNWG-r42 |

Secretogranin 5

| | | | | |
|---|---|---|---|---|
| 58 | 181-202 | 2448.3 | — | SVNPYLQGQRLDNVVAKKS |
| | | | | VPH |
| 59 | 181-211 | 3510.7 | with and | SVNPYLQGQRLDNVVAKKS |
| | | 3590.7 | without | VPHFSDEDKDPE |
| | | | phos | |
| 60 | 199-211 | 1500.6 | - | SVPHFSDEDKDPE |

Secretogranin 2

| | | | | |
|---|---|---|---|---|
| 61 | 529-566 | 4152.9 | | GQGSSEDDLQEEEQIEQAI |
| | | | | KEHLNQGSSQETDKLAPVS |
| 62 | 591-610 | 2385.2 | - | MKVLEYLNQEKAEKGREHI |
| | | | | A |
| 63 | 285-312 | 3086.6 | | SGQLGIQEEDLRKESKDQL |
| | | | | SDDVSKVIA |
| 64 | 569-584 | 1843.9 | | FPVGPPKNDDTPNRQY |
| 65 | 598-610 | 1508.8 | | NQEKAEKGREHIA |
| 66 | 527-564 | 4162.9 | | VPGQGSSEDDLQEEEQIEQ |
| | | | | AIKEHLNQGSSQETDKLAP |
| 67 | 569-586 | 2145.0 | | FPVGPPKNDDTPNRQYWD |
| 68 | 569-587 | 2274.0 | | FPVGPPKNDDTPNRQYWDE |
| 69 | 569-609 | 4911.4 | | FPVGPPKNDDTPNRQYWDE |
| | | | | DLLMKVLEYLNQEKAEKGR |
| | | | | EHI |
| 70 | 569-610 | 4982.5 | | FPVGPPKNDDTPNRQYWDE |
| | | | | DLLMKVLEYLNQEKAEKGR |
| | | | | EHIA |
| 71 | 571-610 | 4738.3 | | VGPPKNDDTPNRQYWDEDL |
| | | | | LMKVLEYLNQEKAEKGREH |
| | | | | IA |

Chromogranin A

| 72 | 105-131 | 2991.4 | — | SEVLENQSSQAELKEAVEE |
|---|---|---|---|---|
| | | | | PSSKDVME |
| 73 | 134-160 | 2899.3 | — | EDSKEAEKSGEATDGARPQ |
| | | | | ALPEPMQE |
| 74 | 134-175 | 4469 | — | EDSKEAEKSGEATDGARPQ |
| | | | | ALPEPMQESKAEGNNQAPG |
| | | | | EEEE |
| 75 | 134-224 | 9653.4 | — | EDSKEAEKSGEATDGARPQ |
| | | | | ALPEPMQESKAEGNNQAPG |
| | | | | EEEEEEEEATNTHPPASLP |
| | | | | SQKYPGPQAEGDSEGLSQG |
| | | | | LVDREKGLSAEPGWQ |
| 76 | 134-225 | 9724.4 | — | EDSKEAEKSGEATDGARPQ |
| | | | | ALPEPMQESKAEGNNQAPG |
| | | | | EEEEEEEEATNTHPPASLP |
| | | | | SQKYPGPQAEGDSEGLSQG |
| | | | | LVDREKGLSAEPGWQA |
| 77 | 135-226 | 9723.5 | — | DSKEAEKSGEATDGARPQA |
| | | | | LPEPMQESKAEGNNQAPGE |
| | | | | EEEEEEEATNTHPPASLPS |
| | | | | QKYPGPQAEGDSEGLSQGL |
| | | | | VDREKGLSAEPGWQAK |
| 78 | 136-189 | 5730.6 | — | SKEAEKSGEATDGARPQAL |
| | | | | PEPMQESKAEGNNQAPGEE |
| | | | | EEEEEEATNTHPPASL |
| 79 | 310-339 | 3388.7 | — | AVVPQGLFRGGKSGELEQE |
| | | | | EERLSKEWEDS |
| 80 | 413-456 | 5061.5 | — | GYPEEKKEEEGSANRRPED |
| | | | | QELESLSAIEAELEKVAHQ |
| | | | | LQALRR |
| 81 | 435-453 | 2065.1 | — | ESLSAIEAELEKVAHQLQA |
| 82 | 435-456 | 2490.4 | — | ESLSAIEAELEKVAHQLQA |
| | | | | LRR |
| 83 | 440-456 | 2003.1 | — | IEAELEKVAHQLQALRR |
| 84 | 441-456 | 1890.1 | — | EAELEKVAHQLQALRR |
| 85 | 97-131 | 3905.8 | — | HSGFEDELSEVLENQSSQA |
| | | | | ELKEAVEEPSSKDVME |
| phos/sul = phosphorylated or sulfated | | | | |
| phos = phosphorylated | | | | |
| ox. = oxidized | | | | |
| pyro-Glu = pyroglutamate modification | | | | |
| C-term. = C-terminal end of the polypeptide chain | | | | |
| * The masses are calculated and reported as monoisotopic theoretical masses | | | | |
| ** The symbol ≧ (greater than or equal to) indicates that the mass of this peptide has at least the stated value but may also be larger. | | | | |
| *** DRES peptides which have phosphorylations or sulfations as modification cannot be distinguished by mass spectrometry because the effect of both modifications is an increase of 80 Dalton in the mass. It is, however, possible to distinguish sulfations from phosphorylations by sequencing, because they occur on different amino acid side chains. | | | | |
| **** r1 represents a sequence which corresponds to the sequence or parts of the sequence of the SG1 peptide from amino acid 99 to 88, where r1 may be between 0 and 12 amino acids long, starting from amino acid 100 of the SG1 protein. Correspondingly, r2 represents the SG1 protein sequence from amino acid 108 to 134 or parts thereof, where r2 may be between 0 and 27 amino acids long, starting from SG1 amino acid 107. The other peptide chains r3 to r42 can be inferred in accordance with this scheme from | | | | |
| # FIG. 1 A to D. | | | | |

Suitable Peptides

The peptides can exist in post-translational or chemical modification forms, thus influencing inter alia their masses and the identification by mass spectrometry and also the elution behaviour on chromatography such as, for example, on reverse phase chromatography. In particular, the peptides may be in phosphorylated, sulfated, N-glycosylated or O-glycosylated form or with N-terminal pyroglutamate modification or in oxidized form etc. in the sample to be investigated.

It is to be assumed that the changes in concentration of the marker peptides correlate with the severity of the disease, the prognosis and the stage of the neurological disease, in particular the progressive, chronic dementia disease, in particular Alzheimer's disease. This is in particular true when a panel of markers as disclosed herein is used. A further embodiment of the invention is therefore to use the determination of the marker peptides also for establishing the severity, the prognosis and for determining the stage of the disease, in particular as substitute or as supplement to carrying out a mini-mental state examination (MMSE) or other neuropsychological investigations. A further development of the invention additionally provides for using determination of the marker peptides to identify preliminary stages of neurological diseases, in particular mild cognitive impairment (MCI), or for the prognosis of the course of the disease.

The control samples which are possibly used may be a pooled sample from various controls. The sample to be investigated may also be a pooled sample and, where there is a positive result, individual investigations are carried out.

Suitable Biological Samples

The liquid biological sample may preferably be (human) cerebrospinal fluid (CSF) or a sample such as serum, plasma, urine, whole blood, cells, tissue homogenates, stool, tear fluid, sputum, saliva, synovial fluid etc. This depends inter alia on the sensitivity of the chosen detection method (mass spectrometry, ELISA etc.). Serum, plasma, whole blood, urine, stool, tear fluid and saliva are of particular interest because this sample material is obtained frequently and without great effort from patients during standard investigations.

It is also possible to use homogenized tissue samples obtained, for example, from biopsy specimens. It is therefore provided in a further embodiment of this invention for tissue homogenates to be produced, for example from human tissue samples obtained in biopsies, for preparation of the sample to be investigated. These tissues can be comminuted for example with manual homogenizers, with ultrasound homogenizers or with electrically operated homogenizers such as, for example, Ultraturrax, and subsequently be boiled in a manner known to the skilled worker in acidic aqueous solutions with, for example, 0.1 to 0.2 M acetic acid for 10 minutes. The extracts are then subjected to the respective detection method, e.g. a mass spectrometric investigation. The samples can be prepared, for example where appropriate diluted or concentrated, and stored in the usual way.

Use of SG1 Proteins. SG1 Peptides and DRES Peptides for Producing Diagnostics

The invention further comprises the use of at least one DRES peptide, SG1 peptide or SG1 protein for the diagnosis of neurological diseases, in particular chronic dementia diseases, in particular Alzheimer's disease, and the use of DRES peptides and SG1 peptides for obtaining antibodies or other agents which, because of their specific binding properties, are suitable for developing diagnostic reagents for detecting these diseases.

Detection Methods for SG1 Protein, SG1 Peptide or DRES Peptides

Various methods can be used for detecting SG1 proteins, SG1 peptides or DRES peptides within the framework of the invention. Methods suitable for this are all those which make it possible to detect these substances specifically in a patient's sample. Suitable methods are, inter alia, physical methods such as, for example, mass spectrometry or liquid chromatography, molecular biology methods such as, for example, reverse transcriptase polymerase chain reaction (RT-PCR) or immunological detection techniques such as, for example, enzyme-linked immunosorbent assays (ELISA), and many other methods known to the skilled worker [2, 3, 14-17].

In a preferred embodiment, at least one peptide derived from chromogranin A, secretogranin 2, and/or secretogranin 5 is detected beside the above described detection of secretogranin 1. Thus, a correlation related network comprising at least two peptides depicted in table 1 from different proteins is used for obtaining an improved diagnostic tool in diagnosing neuronal diseases, in particular chronic dementia diseases, like Alzheimer's disease.

A particular preferred embodiment is the detection of the peptides specified in table 2 below. Said peptides represent a marker panel for diagnostic purposes, in particular diagnosing Alzheimer's disease.

Physical Detection Methods

One embodiment of the invention is the use of physical methods which are able to indicate the peptides of the invention qualitatively or quantitatively [2, 3, 14-17]. These methods include, inter alia, methods such as liquid chromatography, thin-layer chromatography, circular dichroism (CD spectroscopy), biochip technologies using nucleic acids, proteins, antibodies etc. (Ciphergen Biosystems, Inc., Fremont, Calif., USA), and many different spectroscopic methods which operate, for example, with electromagnetic radiation in various wavelength ranges (e.g. wavelengths from 1 nm to 1 m). These methods include, for example, atomic spectroscopy, chemiluminescence spectroscopy, electron spectroscopy, X-ray spectroscopy, infrared spectroscopy, Fourier transform IR spectroscopy, Raman spectroscopy, laser spectroscopy, hole-burning spectroscopy, luminescence spectroscopy, plasma spectroscopy, magnetic resonance spectroscopy (NMR), mass spectroscopy, microwave spectroscopy, Mössbauer spectroscopy, fluorescence spectroscopy, UV/visible spectroscopy etc. This entails comparison of quantitative measured results from a sample to be investigated with the measurements obtained from a group of patients suffering from neurological diseases, in particular chronic dementia diseases, preferably Alzheimer's disease, and a control group. It is possible to infer the presence of a neurological disease, in particular a chronic dementia disease, in particular Alzheimer's disease, and/or the severity and/or a prognosis of this disease from these results.

In a preferred embodiment of this invention, the peptides in the sample are separated by chromatography before the determination, in particular preferably by reverse phase chromatography, with particular preference for separation of the peptides in the sample by high-resolution reverse phase high-performance liquid chromatography (RP-HPLC). A further embodiment of this invention is the carrying out of precipitation reactions to fractionate the sample using precipitants such as, for example, ammonium sulfate, polyethylene glycol, trichloroacetic acid, acetone, ethanol etc. Other precipitation methods such as, for example, immunoprecipitation with antibodies or precipitation reactions induced by, for example, changing physical factors such as temperature (heat precipitation) or pressure can also be used. The fractions obtained in this way are then subjected singly to the respective detection method, e.g. the mass spectrometric investigation. A further embodiment of the invention is the use of extraction methods such as, for example, liquid phase extraction. For this purpose, the sample is mixed for example with a mixture of an organic solvent such as, for example, polyethylene glycol (PEG) and an aqueous salt solution. Owing to their physical properties, particular constituents of the sample then accumulate in the organic phase, and others in the aqueous phase, and can thus be separated from one another and subsequently analyzed further.

Reverse Phase Chromatography

A particularly preferred embodiment of this invention encompasses the use of reverse phase chromatography, in particular a C18 reverse phase chromatography column, using mobile phases consisting of trifluoroacetic acid and acetonitrile, for separating peptides in human cerebrospinal fluid. For example the fractions collected in each case each comprise 1/100 of the volume of mobile phase used. The fractions obtained in this way are analyzed with the aid of a mass spectrometer, preferably with the aid of a MALDI mass spectrometer (matrix-assisted laser desorption ionization) using a matrix solution consisting of, for example, L(-) fucose and alpha-cyano-4-hydroxycinnamic acid dissolved in a mixture of acetonitrile, water, trifluoroacetic acid and acetone, and thus the presence of particular masses is established and the signal intensity is quantified. These masses correspond to the masses of e.g. the peptides DRES-1 to DRES-45 of the invention.

Mass Spectrometry

In a preferred embodiment of the invention, identification of the peptide(s) can be carried out with the aid of a mass spectrometric determination, preferably a MALDI-TOF (Matrix-assisted laser desorption and ionization time of flight) mass spectrometry. In this case, the mass spectrometric determination further preferably includes at least one of the following mass signals, in each case calculated on the basis of the theoretical monoisotopic mass of the corresponding peptide. It is possible for slight differences from the theoretical monoisotopic mass to occur owing to a small measurement inaccuracy of the mass spectrometer not exceeding 500 ppm and the natural isotope distribution. In addition, in MALDI mass determination a proton is added to the peptides owing to the method of measurement, whereby the mass increases by 1 Da. The following masses correspond to the theoretical monoisotopic masses of the peptides identified by us, calculated with suitable software, in this case GPMAW 4.02. These theoretical monoisotopic masses may occur singly or in combinations in a sample: DRES-1=4605.0/DRES-2=4620.1/DRES-3=4392.9/DRES-4=4107.8/DRES-5=4321.9/DRES-6=2853.3/DRES-7=2368.1/DRES-8=4619.0/DRES-9 =4335.9/DRES-10=3246.5/DRES-11=≧686.3/DRES-12≧934.4/DRES-13≧891.4/DRES-14=6433.7/DRES-15=4583.1/DRES-16=4427.0/DRES-17=2522.1/DRES-18≧835.4/DRES-19≧805.3/DRES-20≧864.3/DRES-21=3202.4 /DRES-22≧774.3/DRES-23≧933.4/DRES-24=1985.8/DRES-25≧991.4/DRES-26=1992.8/DRES-27≧976.4/DRES-28=4750.2/DRES-29≧906.3/DRES-30≧892.5/DRES-31=6499.0/DRES-32=6264.9/DRES-33=5565.6/DRES-34=5067.3/DRES-35=4867.2/DRES-36=4791.2/DRES-37≧835.3/DRES-38≧930.4/DRES-39≧1001.4/DRES-40=6970.3/DRES-41≧862.4/DRES-42=1588.9/DRES-43≧919.4 and DRES-44≧942.5, DRES-45=1288,6, DRES-46=1677,8, DRES-48=1268,6, DRES-49=1422,7, DRES-50=1096,5, DRES-52=1341,5, DRES-54=1023,6, DRES-55=1233,7, peptides derived from Chromogranin A (see table 1): 2991,4 2899,3/4469/9653,4/9724,4/9723,5/5730,6/5061,5/ 2065,1/2490,4 2003,1/1890,1/3905,8; peptides derived from secretogranin 2: 1219,6 4152,9 2385,2 3100,5 1829,9 1508,7 4180 2030 2159,1 4796,4 4867,4 4657,3/3086,6/1843,9/1508,8/4162,9 /2145,0 2274,0/4911,4/4982,5/4738,3/DRES-47≧975,5 DRES-51≧1111,5, DRES-53≧952,4; peptides derived from secretogranin 5: 3510,7/ 1500,6/2448,3 or 3590,7 Dalton. The symbol ≧ (is greater than or equal to) is to be understood to mean here that the relevant DRES peptide cannot have arbitrary larger masses but can have only the masses which result owing to the amino acids which are possibly additionally present at the ends of these peptides. Amino acids which may be additionally present at the ends of these peptides are not just any ones but only those which may be present at this sequence position owing to the sequence of an SG1 protein. In addition, it was possible to identify and determine experimentally peptide variants having 0, 1, 2 and 3 phosphate groups/sulfate groups for DRES-14. DRES-15, DRES-16 and DRES-36 occur with and without a phosphate/sulfate group. In addition, DRES-21 has been determined as peptide oxide (oxidation at the Met318 position) and as phosphorylated/sulfated peptide oxide (oxidation at the Met381 position, phosphate/sulfate group at the Ser311 position). DRES-32 has been found with an N-terminal pyroglutamate modification and DRES-42 with a C-terminal amidation.

With respect to the peptides derived from chromogranin A, secretogranin 2 and secretogranin 5 reference is made to table 1 above.

Mass Spectrometric Determination of the Sequence of DRES Peptides

For the further practical application of this embodiment, further confirmation of the result of detection is advisable and possible by establishing the identity of the peptides corresponding to the masses, taking account exclusively of peptide signals which can be derived from an SG1 protein. This confirmation takes place by identifying the peptide signals preferably by mass spectrometric methods, e.g. an MS/MS analysis [18].

Novel, specific peptides derived from secretogranin 1 have been identified and their significance has been recognized. These peptides and their derivatives are referred to herein as DRES-1 to DRES-45. Their sequences are indicated in the sequence listing. The DRES peptides DRES-11, -12, -13, -18, -19, -20, -22, -23, -25, -27, -29, -30, -37, -38, -39, -43, -44, -47, -51, and 53 may comprise at the N and/or C terminus additional amino acids corresponding to the corresponding sequence of secretogranin 1. The invention also encompasses DRES peptides, SG1 peptides and SG1 proteins which have been produced recombinantly, enzymatically or synthetically and isolated from is biological samples and which are in unmodified, chemically, enzymatically or post-translationally modified form.

Further, the invention provides novel peptides derived from chromogranin A, secretogranin 2 and secretogranin 5, respectively. The particular preferred peptides are depicted in table 1 above. Said peptides may be produced recombinantly, enzymatically or synthetically or may be isolated from biological samples and may be in unmodified, chemically, enzymatically or post-translationally modified form. Further within the scope of the present invention are peptides having at least 70% homology with the peptides as defined above.

Molecular Biology Detection Techniques

Finally, the invention also encompasses nucleic acids which correspond to DRES peptides, and especially those which correspond to the DRES peptides of the invention, and the use thereof for the indirect determination and quantification of the relevant SG1 and DRES peptides. This also includes nucleic acids which, for example, represent noncoding sequences such as, for example, 5′- or 3′-untranslated regions of the mRNA, and nucleic acids which show a sequence agreement, sufficient for specific hybridization experiments, with a nucleic acid sequence of SG1 proteins, and which are therefore suitable for indirect detection of the relevant SG1 proteins, SG1 peptides, especially of the DRES peptides.

One exemplary embodiment thereof encompasses the obtaining of tissue samples, e.g. of biopsy specimens, from patients and subsequent determination of the concentration of an RNA transcript corresponding to the sequence having the GenBank Accesion No. NM_{—}001819 or corresponding to sequences having at least 70% homology to NM_{—}001819. This entails comparison of quantitative measured results (intensities) from a sample to be investigated with the measurements obtained in a group of patients suffering from Alzheimer's disease and a control group. Methods which can be used for the quantification are, for example, reverse transcriptase polymerase chain reaction (RT-PCR), quantitative real-time PCR (ABI PRISM® 7700 Sequence Detection System, Applied Biosystems, Foster City, Calif., USA), Northern blots etc., and other methods known to the skilled worker [2, 3, 14-17]. The presence of a neurological disease, preferably a chronic dementia disease, preferably Alzheimer's disease, and/or the severity thereof and/or a prognosis for the occurrence of the disease can be inferred from the results.

Immunological Detection Methods

In a further preferred embodiment of the invention, the determination of the peptides or proteins according to the present invention can be carried out using an immunological detection system, preferably an ELISA (enzyme-linked immunosorbent assay). This immunological detection picks up at least one SG1 protein, one SG1 peptide or one DRES peptide. To increase the specificity, it is also possible and preferred to use a so-called sandwich ELISA in which the detection of the DRES peptides, SG1 peptides and SG1 proteins depends on the specificity of two antibodies which recognize different epitopes within the same molecule. However, it is also possible to use other ELISA systems, e.g. direct or competitive ELISA, to detect these substances. Other ELISA-like detection techniques such as, for example, RIA (radio-immunoassay), EIA (enzyme immunoassay), ELI spot etc. are also suitable as immunological detection systems. DRES peptides, SG1 peptides or SG1 proteins which have been isolated from biological samples, produced recombinantly or enzymatically or synthesized chemically can be used as standard for the quantification. Determination of the DRES peptide(s), SG1 peptide(s) or SG1 protein(s) is generally possible for example with the aid of an antibody directed to the lo particular substances. Further methods suitable for such detections are, inter alia, Western blotting, immunoprecipitations, dot-blots, plasmon resonance spectrometry (BIACORE® technology, Biacore International AB, Uppsala, Sweden), affinity matrices (e.g. ABICAP technology, ABION Gesellschaft für Biowissenschaften und Technik mbH, Jülich, Germany) etc. Substances/molecules suitable as detection agents are generally all those permitting the construction of a specific detection system because they specifically bind a DRES peptide, SG1 peptide or SG1 protein. Numerous immunological detection methods known to the skilled worker but not expressly mentioned here are likewise suitable for this [3, 15]. According to a preferred embodiment, the markers derived from chromogranin A, secretogranin 2 and/or secretogranin 5, as described herein, are additionally detected with the above mentioned methods.

Obtaining SG1 Proteins, SG1 Peptides, DRES Peptides and the Peptides Derived Form Secretogranin 2, Secretogranin 5 and Chromogranin A as Specified in Table 1

A further embodiment of the invention is the production of DRES peptides, SG1 peptides, SG1 proteins and the other peptides specified in table 1 using recombinant expression systems, in vitro translation, chromatographic methods and chemical synthesis protocols etc., which are known to the skilled worker. These substances can be obtained from natural biological samples or from recombinant expression systems, for example using reverse phase chromatography, affinity chromatography, ion exchange chromatography, gel filtration, isoelectric focussing, preparative immunoprecipitation, ammonium sulfate precipitation, extraction with organic solvents etc., and with other methods known to the skilled worker. The substances obtained in this way can be used inter alia as therapeutic agent for treating neurological diseases, in particular Alzheimer's disease, as standards for quantifying the respective peptides or as antigen for producing antibodies. Said peptides or proteins may be C— or N-terminally fused to heterologous sequences from foreign peptides such as polyhistidine sequences, hemagglutinin epitopes (HA tag), or proteins such as, for example, maltose-binding proteins, glutathione S-transferase (GST), or protein domains such as the GAL-4 DNA binding domain or the GAL4 activation domain 12, 3, 15].

Obtaining Antibodies Directed Towards the Peptides According to Table 1

A further preferred embodiment of the invention is the production and obtaining of antibodies directed towards the peptides disclosed in table 1, in particular to DRES peptide-specific antibodies, and a particularly preferred embodiment is the production of DRES peptide-specific antibodies which recognize new epitopes, i.e. epitopes which are present only on DRES peptides but not in a peptide which, besides the DRES peptide sequence, also comprises other sequences. Such specific peptide antibodies make the specific immunological detection of the peptides possible in the presence of the whole protein, e.g. secretogranin 1. Polyclonal antibodies can be produced by immunizations of experimental animals such as, for example, mice, rats, rabbits or goats. Monoclonal antibodies can be obtained for example by immunizations of experimental animals such as, for example, mice or rats and subsequent use of hybridoma techniques or else via recombinant experimental approaches such as, for example, via antibody libraries such as the HuCAL® antibody library of MorphoSys, Martinsried, Germany, or other recombinant production methods known to the skilled worker. DRES peptide-specific antibodies can also be used in the form of antigen-binding antibody fragments. Examples of such antibody fragments are intrabodies, fab (fragment, antigen binding), F(ab′)₂ or scFv (single-chain Fv fragment) fragments. The antibodies can also be produced recombinantly or synthetically as fusion proteins consisting of one or more antibody proteins or antibody protein fragments and one or more other proteins or protein fragments, such as, for example, enzymes, fluorescent proteins etc. [2, 3, 15].

Therapy Development and Monitoring

A further embodiment of the invention is the quantitative or qualitative measurement of the abovementioned peptides and proteins, for example the DRES peptides, SG1 peptides or SG1 proteins for estimating the efficacy of a therapy under development for neurological diseases, in particular chronic dementia diseases, in particular Alzheimer's disease. The invention can also be is used to stratify participants in clinical studies for the development of therapies for these diseases, especially Alzheimer's disease. The testing of efficacy and the selection of the correct patients for therapies and for clinical studies is of outstanding importance for successful development and application of a therapeutic agent. No clinically measurable parameter making this reliably possible is yet available for Alzheimer's disease [19].

Examination of the Therapeutic Efficacy of Proteins and Peptides According the Present Invention and of Agents Which Modulate the Expression and Bioavailability of these Substances

One exemplary embodiment thereof encompasses the cultivation of cell lines and their treatment with SG1 proteins, SG1 peptides or DRES peptides or with substances which promote the expression or processing of these compounds. Substances which promote processing may be, for example, proteases such as prohormone convertases which recognize “dibasic sequence motifs”. It is possible thereby to establish possible therapeutic uses of SG1 proteins, SG1 peptides and DRES peptides in connection with neurological diseases, in particular Alzheimer's disease. Fusion proteins can also be used for treating the cell lines, such as, for example, fusion proteins having peptide sequences which promote transport of the fusion protein into the interior of the cell. Examples of possible fusion partners are HIV TAT, antennapedia, herpes simplex VP22 sequences etc. It is likewise possible to transfect cell lines with expression vectors which influence, directly or indirectly, the expression of SG1 proteins, SG1 peptides or DRES peptides by the transfected cells, e.g. by coding directly for these substances or by coding for prohormone convertases, expression factors etc. which are involved in the processing or expression of these substances. Simultaneous transfection with a plurality of different expression vectors can also be carried out. Alternatively, suitable cell lines can be treated with anti-SG1 protein, anti-SG1 peptide or anti-DRES peptide antibodies or with nucleic acids which suppress expression of SG1 proteins, SG1 peptides or DRES peptides, such as, for example, SG1 antisense, SG1 triplex, SG1 RNAi nucleic acids or ribozymes directed against secretogranin 1-RNA. Cell lines which appear suitable as neurological model systems in connection with secretogranin 1 can be used in particular for such investigations. Read-out systems which can be used for these investigations are, inter alia, tests which measure the rate of proliferation of the treated cells, their metabolic activity, the rate of apoptosis of the cells, changes in cell morphology, changes in the expression of cell-intrinsic proteins or of reporter genes added to the cells, or which determine the release of cytosolic cell constituents as markers of cell deaths.

Further test systems which can be used are suitable strains of experimental animals, e.g. of mice or rats, which are regarded as a model of neurological diseases, in particular as a model of Alzheimer's disease. These experimental animals can be used to investigate the efficacy of therapeutic strategies which aim to modulate the concentration of said peptides or proteins. It is additionally possible to investigate the in vivo effect of these substances in suitable experimental animals such as, for example, mice, rats, rabbits, dogs, monkeys etc.

Parameters measured in experiments with experimental animals may be, for example, the survival time of the animals, their behavior, their short-term memory and their learning ability. One example of a memory test suitable for experimental animals such as, for example, rats is the Morris water maze test. Further parameters which can be used are the determination of body function (temperature, breathing rate, heart rate, etc.), the determination of, for example, neurological mediators from, for example, blood, urine, tissue samples or CSF, measurement of brain currents, metabolic tests, the expression of SG1 proteins, SG1 peptides or DRES peptides and other peptides connected with the disease, e.g. as exemplified in table 1, and morphological and histological investigations on tissues such as, for example, the brain.

A further possibility for investigating the therapeutic efficacy of the proteins and peptides according to the present invention is the possibility of obtaining by methods of molecular biology experimental animals in whose organism these substances are not produced, or are produced in a reduced or increased amount. It is possible in this way for expression to be changed in a targeted manner, both locally and in the whole organism of the experimental animal. Suitable experimental animals are, inter alia, Caenorhabditis elegans, drosophila, zebra fish, mice, rats etc.

Screening Methods

A further embodiment of the invention relates to methods for finding substances which modulate the expression, concentration or activity of the proteins or peptides according to the present invention or of nucleic acids which code therefore. The invention includes in particular methods in which a sample which comprises at least one protein or peptide according to the present invention or a corresponding nucleic acid is brought into association with a test substance. These methods investigate whether the test substance has the ability to modulate the expression of said proteins or peptides, e.g. of the proteins and peptides derived from secretogranin 1, or whether the test substance influences the activity or concentration of said proteins or peptides.

The invention is illustrated in detail below by means of examples. Reference is also made to the figures in this connection.

FIG. 1: Alignment of the DRES peptides with secretogranin 1

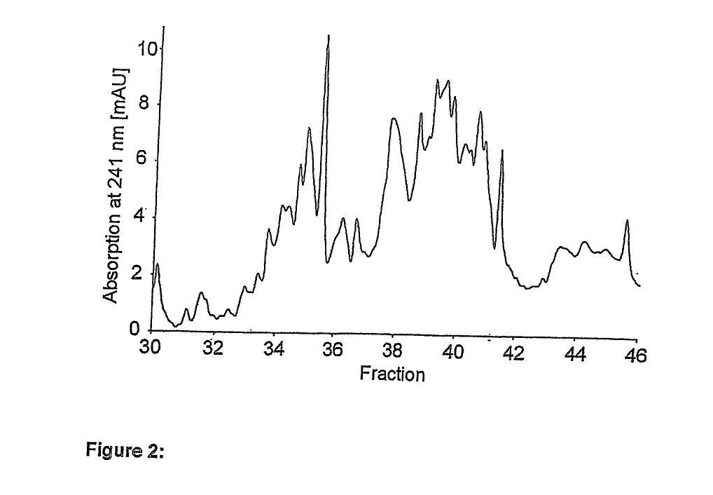

FIG. 2: Reverse phase chromatography for separation and concentration of the DRES peptides from cerebrospinal fluid

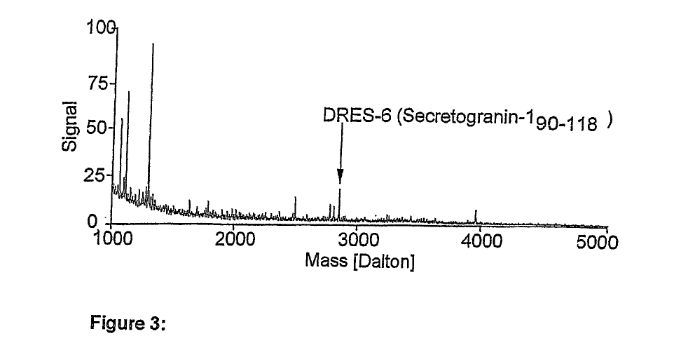

FIG. 3: Mass spectrometric measurement (MALDI) on DRES-6 as example

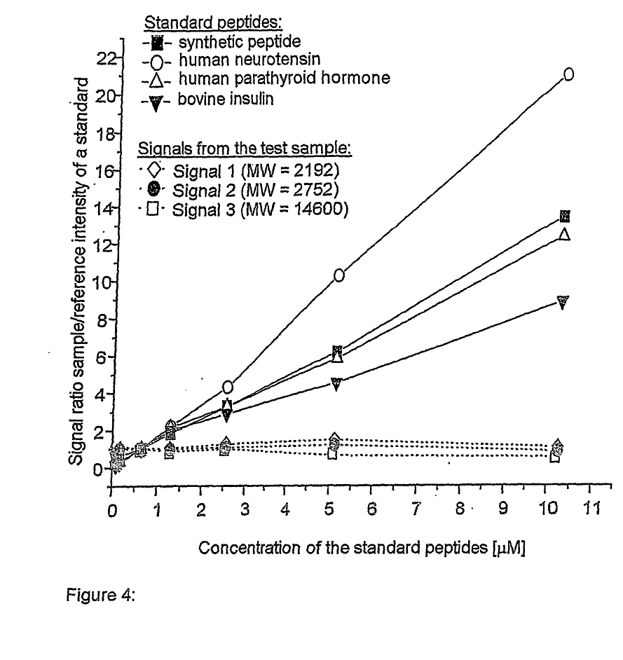

FIG. 4: MALDI as relatively quantifying mass spectroscopic method

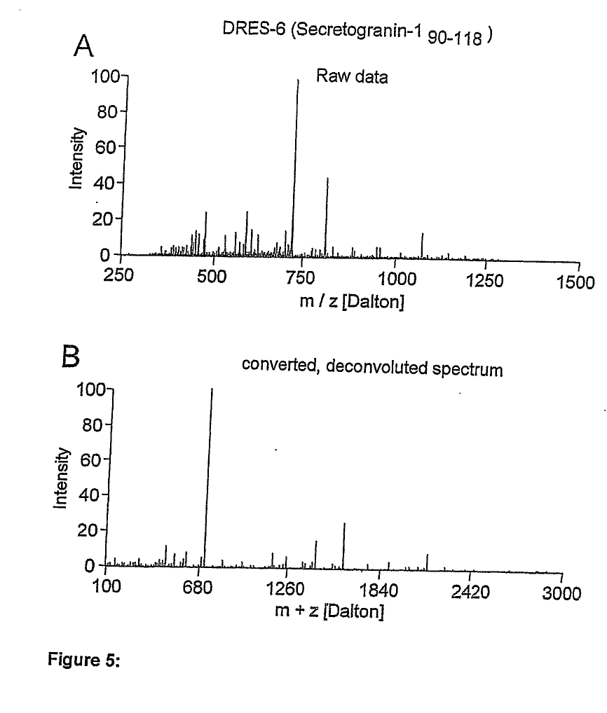

FIG. 5: MS/MS fragment spectrum on the DRES-6 peptide as example

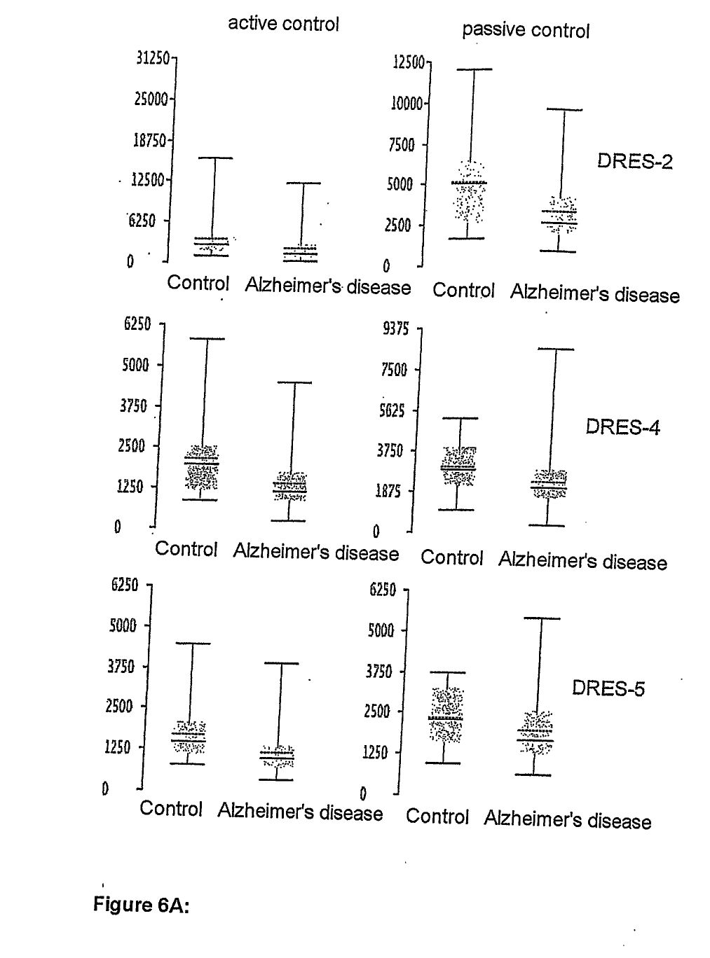

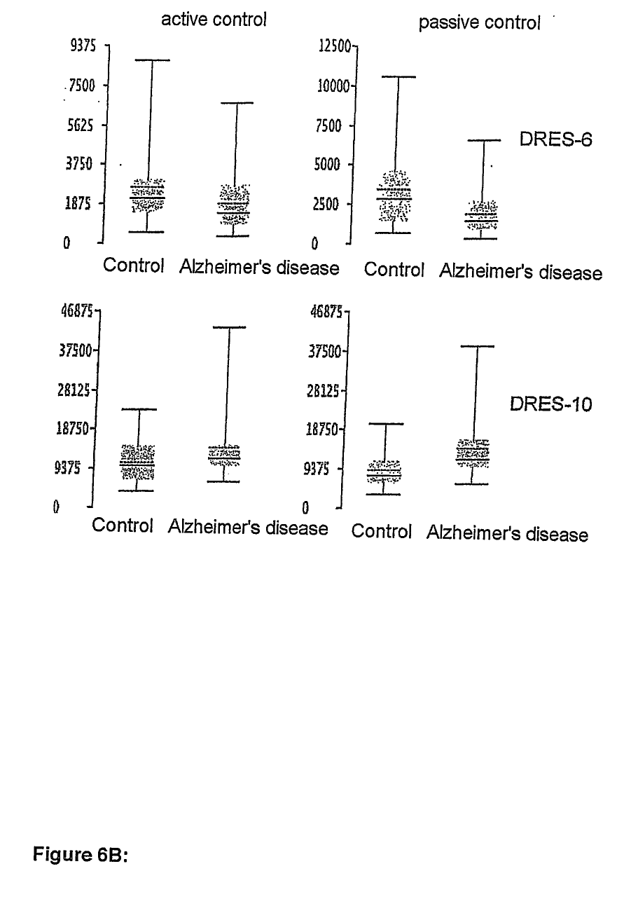

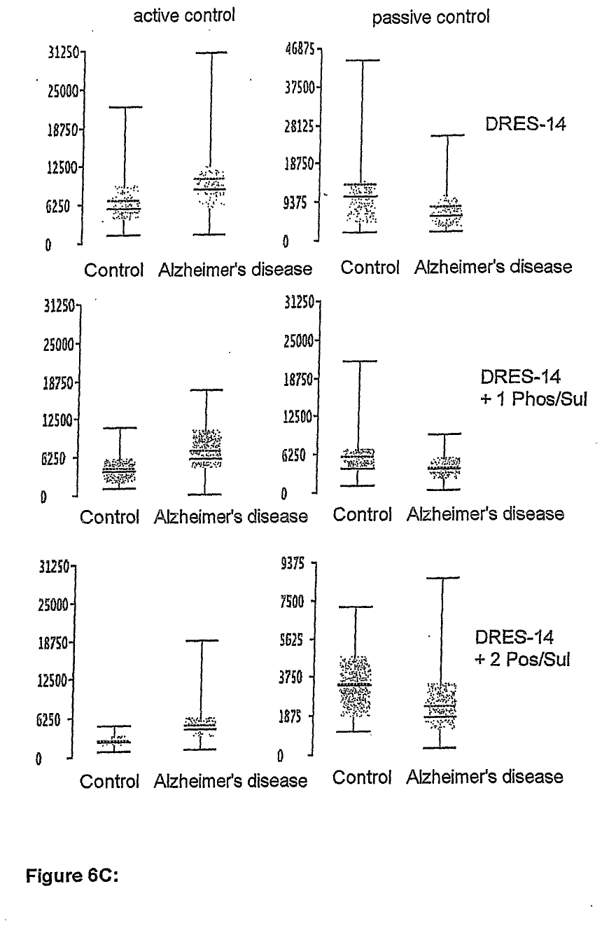

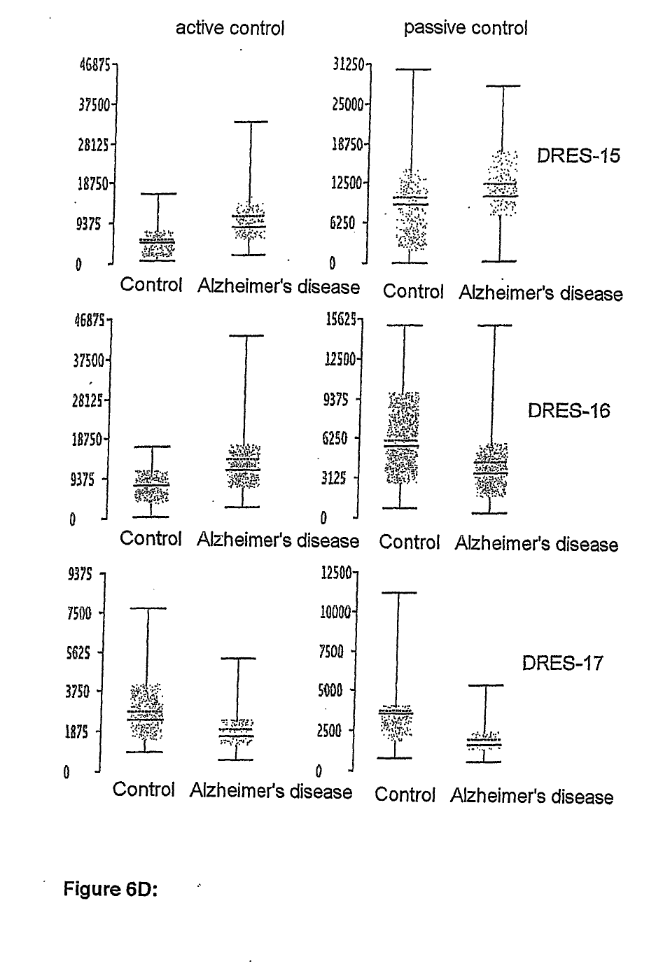

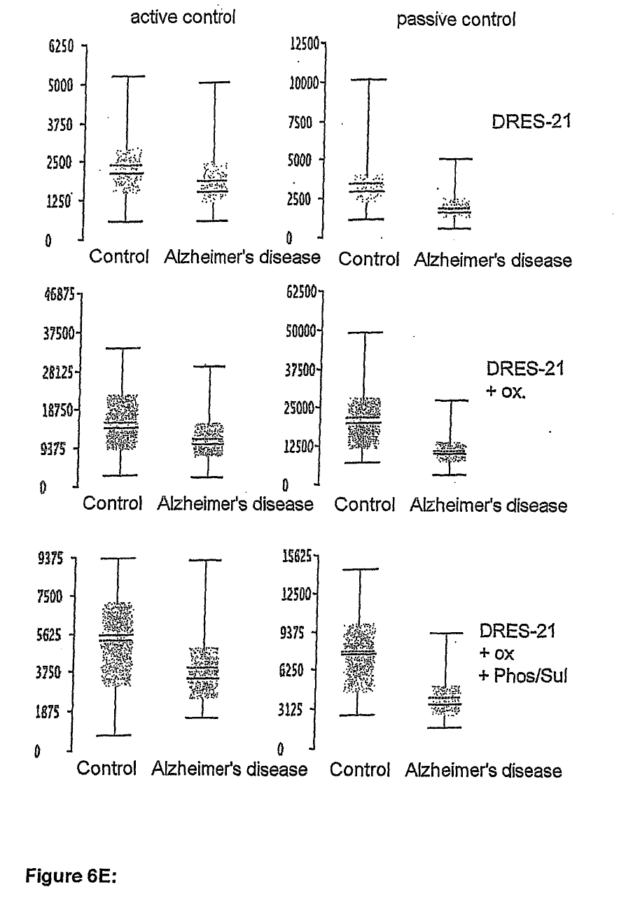

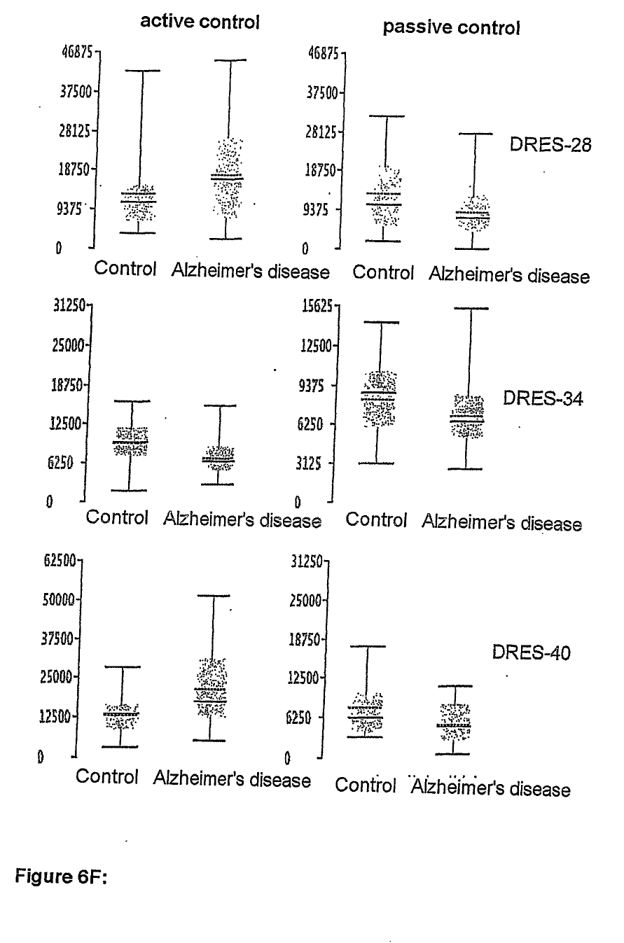

FIG. 6A-6F: Box-whisker plots for quantitative comparison of the concentrations of DRES-2, -4, -5, -6, -10, -14, -15, -16, -17, -21, -28, -34 and DRES-40 in patients with Alzheimer's disease compared with control patients (ox.=oxidized peptide, phos/sul=phosphorylated or sulfated peptide).

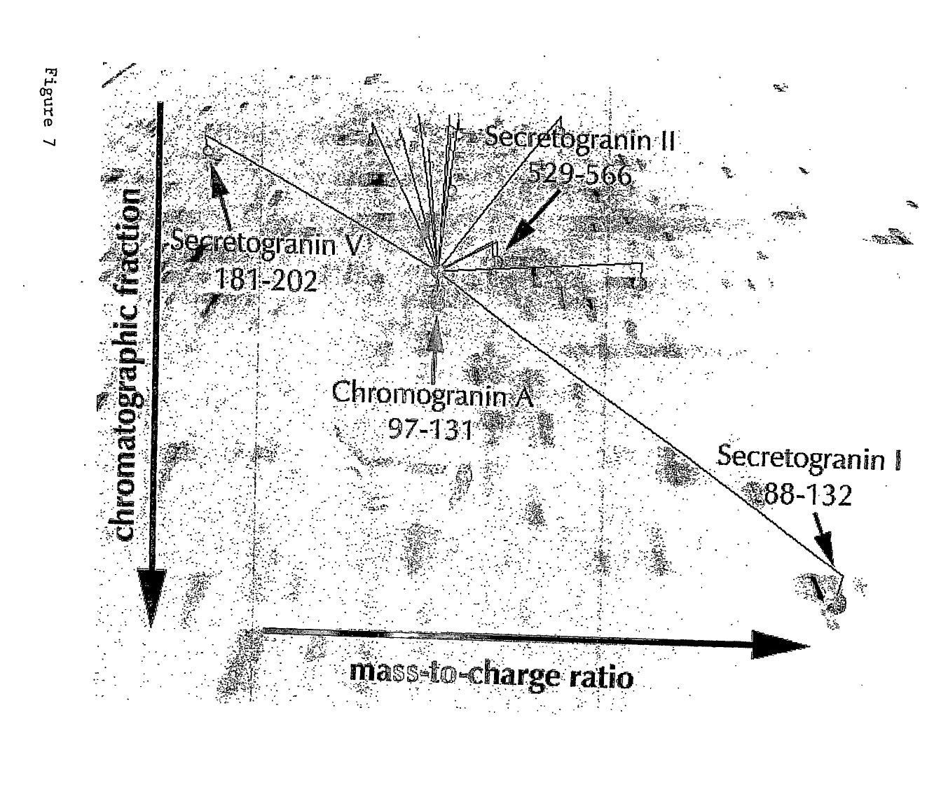

FIG. 7: Correlation-associated network diagramm.

FIG. 1 shows an alignment of the DRES peptides of the invention with secretogranin 1.

FIG. 2 shows an elution profile of a reverse phase chromatography as in Example 2 for separation and concentration of the DRES peptides from cerebrospinal fluid.

FIG. 3 shows a spectrum produced by MALDI mass spectrometric measurement of DRES-6 as in Example 3 after reverse phase chromatography of human cerebrospinal fluid as in Example 2. DRES-6 corresponds to the SG1 sequence from amino acid 90-118.

FIG. 4 shows data generated by MALDI as relatively quantifying MS method. A sample was mixed with various amounts of various standard peptides and the intensity both of these standard signals and of representative sample signals was measured. All signal intensities of the standards were standardized to their signal intensity at a concentration of 0.64 μm (=1). Each peptide shows an individual typical ratio of signal strength to concentration, which can be read off in this diagram from the gradient of the plot. MW=relative molecular mass.

FIG. 5 shows an MS/MS fragment spectrum as in Example 4 of the DRES-6 peptide of the invention.

Upper trace: Raw data of the measurement.

Lower trace: Converted, deconvoluted mass spectrum of DRES-6. DRES-6 corresponds to the secretogranin 1 sequence from amino acid 90 to 118.

FIG. 6 shows box-whisker plots for quantitative comparison of the concentrations of DRES-2, -4, -5, -6, -10, -14, -15, -16, -17, -21, -28, -34 and DRES-40 in patients with Alzheimer's disease compared with control patients, showing for DRES-14 the data of the unmodified peptides (FIG. 6C, top), of the monophosphorylated/sulfated peptides (FIG. 6C, middle) and of the diphosphorylated/sulfated peptides (FIG. 6C, bottom) and for DRES-21 the data of the unmodified peptides (FIG. 6E, top), of the monooxidized peptides (FIG. 6E, middle) and of the monooxidized and simultaneously monophosphorylated/sulfated peptides (FIG. 6E, bottom). The figures show in the form of box-whisker plots a comparison of the integrated MALDI mass spectrometric signal intensities. The left side of FIGS. 6A to E shows in each case the results obtained on comparison of Alzheimer's disease samples with samples from patients with other dementias (active control). The right side of FIGS. 6A to E shows in each case the results obtained on comparison of Alzheimer's disease samples with samples from healthy people of the same age (passive control).

FIG. 7 shows the correlation-associated network automatically depicting peptide signals highly correlating with chromogranin A 97-131 (SEQ ID 85). The network inicudes a secretogranin 1 88-132 (SEQ ID 1), secretogranin II 529-566 (SEQ ID 61) and secretogranin V 181-202 (SEQ ID 58). Correlation threshold is /r/≧0.67.

### EXAMPLE 1

### Obtaining Cerebrospinal Fluid for Determining Pegtides

CSF or cerebrospinal fluid (fluid of the brain and spinal cord) is the fluid which is present in the four ventricles of the brain and in the subarachnoid space and which is produced in particular in the choroid plexus of the lateral ventricle. Cerebrospinal fluid is usually taken by lumbar puncture and less often by suboccipital puncture or ventricular puncture. In lumbar puncture (spinal puncture), to take cerebrospinal fluid, the puncture involves penetration of the spinal subarachnoid space between the 3rd and 4th or the 4th and 5th lumbar spinous process with a long hollow needle, and thus CSF being obtained. The sample is then centrifuged at 2000×g for 10 minutes, and the supernatant is stored at −80° C.

### EXAMPLE 2

### Separation of Peptides in Cerebrospinal Fluid (CSF) for Mass Spectrometric Measurement of Pertides

For the detection of DRES peptides in CSF by mass spectrometry, it is necessary in this example to separate the peptide constituents. This sample pretreatment serves to concentrate the peptides of the invention and to remove components which may interfere with the measurement. The separation method carried out is a reverse phase chromatography. Various RP chromatography resins and eluents are equally suitable for this. The separation of the peptides using a C18 reverse phase chromatography column with the size of 4 mm×250 mm supplied by Vydac is described by way of example below. Mobile phases of the following composition were used: mobile phase A: 0.06% (v/v) trifluoroacetic acid, mobile phase B: 0.05% (v/v) trifluoroacetic acid, 80% (v/v) acetonitrile. Chromatography took place at 33° C. using an HP ChemStation 1100 supplied by Agilent Technologies with a micro flow cell supplied by Agilent Technologies. Human cerebrospinal fluid was used as sample. 440 μl of CSF were diluted with water to 1650 μl, the pH was adjusted to 2-3, the sample was centrifuged at 18 000×g for 10 minutes and finally 1500 μl of the sample prepared in this way were loaded onto the chromatography column. The chromatography conditions were as follows: 5% mobile phase B at time 0 min, from time 1 to 45 min continuous increase in the mobile phase B concentration to 50%, from time 45 to 49 min continuous increase in the mobile phase B concentration to 100% and subsequently up to time 53 min constant 100% buffer B. Collection of 96 fractions each of 0.5 ml starts 10 minutes after the start of the chromatography. The chromatogram of a cerebrospinal fluid sample prepared under the experimental conditions described herein is depicted in FIG. 2.

### EXAMPLE 3

### Measurement of Masses of Peptides by Means of MALDI Mass Spectrometry

For mass analysis, typical positive ion spectra of peptides are produced in a MALDI-TOF mass spectrometer (matrix-assisted laser desorption ionization).

Suitable MALDI-TOF mass spectrometers are manufactured by PerSeptive Biosystems Framingham (Voyager-DE, Voyager-DE PRO or Voyager-DE STR) or by Bruker Daltonik Bremen (BIFLEX). The samples are prepared by mixing them with a matrix substance which typically consists of an organic acid. Typical matrix substances suitable for peptides are 3,5-dimethoxy-4-hydroxycinnamic acid, x-cyano-4-hydroxycinnamic acid and 2,5-dihydroxybenzoic acid. A lyophilized equivalent obtained by reverse phase chromatography and corresponding to 500 μl of human cerebrospinal fluid is used to measure the peptides of the invention. The chromatographed sample is dissolved in 15 μl of a matrix solution. This matrix solution contains, for example, 10 g/l α-cyano-4-hydroxycinnamic acid and 10 g/l L(-)fucose dissolved in a solvent mixture consisting of acetonitrile, water, trifluoroacetic acid and acetone in the ratio 49:49:1:1 by volume. 0.3 μl of this solution is transferred to a MALDI carrier plate, and the dried sample is analysed in a Voyager-DE STR MALDI mass spectrometer from PerSeptive Biosystems. The measurement takes place in linear mode with delayed extraction™. An example of a measurement of one of the DRES peptides of the invention is shown in FIG. 3.

The MALDI-TOF mass spectrometry can be employed to quantify peptides such as, for example, the DRES peptides of the invention if these peptides are present in a concentration which is within the dynamic measurement range of the mass spectrometer, thus avoiding detector saturation. This is the case for the measurement of the peptides of the invention in cerebrospinal fluid at a CSF equivalent concentration of 33.3 μl per μl of matrix solution. There is a specific ratio between measured signal and concentration for each peptide, which means that the MALDI mass spectrometry can preferably be used for the relative quantification of peptides. This situation is depicted in FIG. 4. If various amounts of different standard peptides are added to a sample, it is possible to measure the intensity both of these standard signals and of the sample signals. FIG. 4 shows by way of example a MALDI measurement as relatively quantifying MS method. All signal intensities of the standards were standardized to their signal intensity at a concentration of 0.64 μM (=1). Each peptide shows an individual, typical ratio of signal strength to concentration, which can be read off from the gradient of the plot.

### EXAMPLE 4

### Mass Spectrometric Identification of Peptides

For quantification of the peptides of the invention it is necessary to ensure that the mass signals to be analysed of peptides in the fractions obtained by reverse phase chromatography of cerebrospinal fluid, as in Example 2, in fact relate to the DRES peptides of the invention.

The peptides of the invention are identified in these fractions for example using is nanoSpray-MS/MS [18]. This entails a DRES peptide ion in the mass spectrometer being selected in the mass spectrometer on the basis of its specific m/z (mass/charge) value in a manner known to the skilled worker. This selected ion is then fragmented by supplying collisional energy with an impinging gas, e.g. helium or nitrogen, and the resulting DRES peptide fragments are detected in the mass spectrometer in an integrated analysis unit, and corresponding m/z values are determined (principle of tandem mass spectrometry) [20]. The fragmentation behaviour of peptides makes unambiguous identification of the DRES peptides of the invention possible when the accuracy of mass is, for example, 50 ppm by the use of computer-assisted search methods [21] in sequence databases into which the sequence of secretogranin 1 has been entered. In this specific case, the mass spectrometric analysis took place with a quadrupole TOF Instrument, QStar-Pulsar model from Applied Biosystems-Sciex, USA. Examples of MS/MS fragment spectra are shown in FIG. 5.

### EXAMPLE 5

### Mass Spectrometric Quantification of DRES Peptides to Compare Their Relative Concentration in Control Samples Compared with Patients' Samples

A sample preparation as in Example 1 and 2 followed by a MALDI measurement of the DRES peptides of the invention as in Example 3 were carried out on 279 clinical samples, i.e. 86 passive control samples, 66 active control samples and 127 samples from patients suffering from Alzheimer's disease. Examples of MALDI signal intensities are depicted in the form of box-whisker plots in FIGS. 6A to 6F. The box-whisker plots depicted in FIG. 6 are based on measurements carried out in each case on 29 to 45 samples from Alzheimer's disease patients, and 13 to 44 control samples per individual experiment. A total of 4 experiments were carried out in the sense of a cross validation. The box-whisker plots depicted make it possible to compare the integrated MALDI mass spectrometric signal intensities of various DRES peptides in controls with is the MALDI signal intensities in samples from Alzheimer's disease patients. In these, the box, i.e. the columns in the diagrams in FIGS. 6A to 6F, in each case includes the range of MALDI signal intensities in which 50% of the respective MALDI signal intensities are found (2^{nd} and 3^{rd} quartiles), and the lines starting from the box and pointing upward and downward (whiskers) indicate the range in which in each case the 25% of measurements which show the highest signal intensities (upper quartile) are found, and in which the 25% of measurements which show the lowest signal intensities (lower quartile) are found. The full line in the columns indicates the median and the broken line in the columns indicates the mean.

### EXAMPLE 6

### Correlation of Petides Derived From Different Proteins in the Diagnosis of Neurological Diseases

Sample Preparation

Human CSF was collected by lumbar puncture from neurological patients without cognitive impairment (n=44) and from patients suffering from dementia such as vascular dementia, Lewy-body dementia, frontotemporal dementia or Parkinson's disease (n=30). All CSF samples were prepared using mild conditions minimizing sample alternation: The fluid was collected without aspiration; samples were centrifuged for 10 min at 2000 g and the supernatant was stored at −80° C. until analysis.

Reversed Phase HPLC

Peptides were separated using reversed-phase (RP) C18 chromatography. CSF was diluted 1:3.75 with water and pH adjusted to 2-3. These samples were loaded onto RP silica columns (250×4 mm column, Vydac, Hesperia, Calif., USA; HP-ChemStation 1100 Agilent Technologies, Palo Alto, Calif., USA). Retained peptides were eluted using an acetonitrile gradient (4 to 80%) in 0.05% trifluoroacetic acid, collected into 96 fractions and lyophilized. Elution was monitored by UV detection. The retention time of major peptide peaks from repeatedly loaded extracts was used to confirm the reproducibility of the method.

MALDI-ToF Mass Spectrometry

After lyophilization, each HPLC fraction was resuspended in a mixture of a-cyano-4-hydroxicinnamic acid (matrix) and L-fucose (co-matrix) in 0.1% acetonitrile/ trifluoroacetic acid (1:1 v/v) and applied to a matrix-assisted laser-desorption/ionization (MALDI) target, followed by ambient temperature air drying. Sample ionization was performed by application of repeated single laser shots over a representative area of the sample spot. The accelerated ions were analysed in a time-of-flight (ToF) mass spectrometer (Voyager-DE STR, Applied Biosystems, Framingham, Mass., USA) in linear mode.

NanoESI-qTOF-MS/MS

Peptides of interest were identified by mass spectrometric sequencing using nanoESI-qTOF-MS/MS (QSTAR pulsar, Sciex, Toronto, Canada) with subsequent protein database searching. The resulting peptide fragment spectra were achieved in the product ion scan mode (spray voltage 950 V, collision energy 20-40 eV). Up to 200 scans per sample were accumulated. Data processing previous to database searching included charge state de-convolution (Bayesian reconstruct tool of the BioAnalyst program package, Sciex) and de-isotoping (customized Analyst QS macro; Sciex). The resulting spectra were saved in MASCOT (Matrix Science, London, UK) generic file format and submitted to the MASCOT search engine. Cascading searches including several posttranslational modifications in Swiss-Prot (Version 41, www.expasy.ch) and MSDB (Version 030212, EBI, Cambridge, UK) were performed by the MASCOT DAEMON client (Version 1.9, Matrix Science). This procedure allows also identification of modified amino acids as well as determination of their position.

Peptide Mass Fingerprints: Visualization of Mass Spectrometric Data

Each of the 96 chromatographic fractions was analysed individually by MALDI-ToF-mass spectrometry and all fractions generated from one sample were visualized in a 2D-like gel format. Thus, each peal<is depicted as a bar with its color intensity corresponding to the intensity of the corresponding MALDI-peak. The x-, y- and z-axis represent mass to charge ratios (m/z), chromatographic fraction and mass spectrometric signal intensity, respectively. Mass intervals range from 1000 to 15.000 m/z ratios (y-axis).

Data Pre-Processing

The processing of the raw data obtained by mass spectrometry is described in detail in EP application EP04000170.3. In brief, data pre-processing of mass spectra was performed applying baseline correction (RAZOR Library 4.0, Spectrum Square Associates, Ithaca, N.Y., USA) in combination with normalization of the mass spectra to a constant integral value. Outlier samples were defined with the aid of principal component analysis (PCA, Pirouette 3.0, Infometrix Inc., WA, USA) considering the five dominant components. Based on this PCA model samples with a Mahalanobis distance (MD) exceeding a critical value of MDcrit>11.5 were not considered for further analysis.

Correlation-Associated Networks

Correlation-associated networks, a detailed description thereof is provided in EP 04000170.3 have two forms of visualization and user interaction: peptide mass correlograms and three-dimensional projections of the network on a peptide mass fingerprint (FIG. 7). For a correlogram, correlations of signal intensities with one single peptide of interest were calculated using Pearson correlational analysis. For the analysis of one single peptide in a complete set of peptide maps, approximately 1.4 million pair wise Pearson correlations were calculated using pre-processed mass spectra. The resulting diagram depicts only signals which have an absolute correlation coefficient larger than an arbitrary defined threshold.

For the analysis of all peptide-to-peptide relations, comprehensive correlations were performed with the signal intensities of 6409 peak positions found to be the most prominent signals in peptide mass maps of all patients. A pair-wise relation of two peptides was rated by Spearman's rank correlation analysis of their respective signal intensities in all samples. Spearman correlational coefficient and respective peptides coordinates. Higher order correlation-associated networks were implemented using any member of a network as a starting point for an additional round of correlational analysis.

The correlation-associated network of the CSF peptide identified as chromogranin A 97-131 contains Secretogranin I 88-132, Secretogranin II 529-566 and a fragment from secretogranin V 181-202 (FIG. 7 and table 2).

TABLE 2

Relative
Mono-
isotopic

| | | | | |
|---|---|---|---|---|
| Hub- | Corre- | Related | mass | Amino Acid |

| | | | | |
|---|---|---|---|---|
| Peptide | lation | peptide | [Da] | Sequence |

| | | |
|---|---|---|
| Chromogranin A 97-131 | 3905.764 | HSGFEDELSEVLE |
| | | NQSSQAELKEAVE |
| | | EPSSKDVME |

| | | | |
|---|---|---|---|
| r = 0.67 | Secretogranin | 4605.025 | DPADASEAHESSS |
| | I 88-132 | | RGEAGAPGEEDIQ |
| | | | GPTKADTEKWAEG |
| | | | GGHSRE |
| r = 0.71 | Secretogranin | 4152.921 | GQGSSEDDLQEEE |
| | II 529-566 | | QIEQAIKEHLNQG |
| | | | SSQETDKLAPVS |
| r = 0.72 | Secretogranin | 2448.334 | SVNPYLQGQRLDN |
| | V 181-202 | | VVAKKSVPH |

The above identified peptides identified by correlation-associated network allows for the provision of improved marker panels useful in the diagnosis of Alzheimer's disease.

The headings in this document are intended merely to provide structure to the text. They are not intended to limit or restrict the matters described. All the examples are intended to characterize the concept of the invention in more detail but are not intended to restrict the equivalence range of the invention.

If a term in this patent is not unambiguously defined or if it should be unknown to the skilled worker in the particular art or if a term cannot be unambiguously defined from the context, the definition of the particular term mentioned in each case in the following standard works applies. If a term is included in more than one of the works cited below with different definitions, then the definition mentioned in the work included first in the following list always applies. The following publications are cited for this purpose:
The Merck Manual of Diagnosis and Therapy [14]
Molecular Cloning—A Laboratory Manual [2]
Current Protocols in Immunology [15]
Current Protocols in Protein Science [3]
Current Protocols in Pharmacology [16]
Current Protocols in Cell Biology 117]

### REFERENCES

1. Clark, C. M., L. Sheppard, G. G. Fillenbaum, D. Galasko, J. C. Morris, E. Koss, R. Mohs, and A. Heyman. 1999. Variability in annual Mini-Mental State Examination score in patients with probable Alzheimer disease: a clinical perspective of data from-the Consortium to Establish a Registry for Alzheimer's Disease. Arch. Neurol. 56:857-62.

2. Sambrook, J., and D. W. Russell. 2001. Molecular Cloning—A Laboratory Manual. Cold Spring Harbor Laboratory Press, Cold Spring Harbor, N.Y., USA, 3rd Edition.

3. Coligan, J. E., B. M. Dunn, H. L. Ploegh, D. W. Speicher, P. T. Wingfield, and (Editors). 2002. Current Protocols in Protein Science. John Wiley & Son, Inc., Hoboken, N.J., USA.

4. Devereux, J., P. Haeberli, and O. Smithies. 1984. A comprehensive set of sequence analysis programs for the VAX. Nucleic Acids Res. 12:387-95.

5. Altschul, S. F., W. Gish, W. Miller, E. W. Myers, and D. J. Lipman. 1990. Basic local alignment search tool. J Mol Biol. 215:403-10.

6. Needleman, S. B., and C. D. Wunsch. 1970. A general method applicable to the search for similarities in the amino acid sequence of two proteins. J Mol Biol. 48:443-53.

7. Henikoff, S., and J. G. Henikoff. 1992. Amino acid substitution matrices from protein blocks. Proc Natl Acad Sci USA. 89:10915-9.

8. Garavelli, J. S., Z. Hou, N. Pattabiraman, and R. M. Stephens. 2001. The RESID Database of protein structure modifications and the NRL-3D Sequence-Structure Database. Nucleic Acids Res. 29:199-201.

9. Huttner, W. B., H. H. Gerdes, and P. Rosa. 1991. The granin (chromatogranin/secretogranin) family. Trends Biochem Sci 16:27-30.

10. O'Connor, D. T., and K. N. Bernstein. 1984. Radioimmunoassay of chromogranin A in plasma as a measure of exocytotic sympathoadrenal activity in normal subjects and patients with pheochromocytoma. N Engl. J Med. 311:764-70.

11. Takiyyuddin, M. A., R. J. Parmer, M. T. Kailasam, J. H. Cervenka, B. Kennedy, M. G. Ziegler, M. C. Lin, J. Li, C. E. Grim, F. A. Wright, et al. 1995. Chromogranin A in human hypertension. Influence of heredity. Hypertension. 26:213-20.

13. Strub, J. M., P. Garcia_Sablone, K. Lonning, L. Taupenot, P. Hubert, A. Van_Dorsselaer, D. Aunis, and M. H. Metz_Boutigue. 1995. Processing of chromogranin B in bovine adrenal medulla. Identification of secretolytin, the endogenous C-terminal fragment of residues 614-626 with antibacterial activity. European Journal of Biochemistry. 229:356-68.

14. Beers, M. H., R. Berkow, and (Editors). 1999. The Merck Manual of Diagnosis and Therapy. Merck Research Laboratories, Whitehous Station, N.J., USA. 17th Edition.

15. Coligan, J. E., A. M. Kruisbeek, D. H. Margulies, E. M. Shevach, W. Strober, and (Editors). 2002. Current Protocols in Immunology. John Wiley & Son, Inc., Hoboken, N.J., USA.

16. Enna, S. J., M. Williams, J. W. Ferkany, T. Kenakin, R. D. Porsolt, J. P. Sullivan, and (Editors). 2002. Current Protocols in Pharmacology. John Wiley & Son, Inc., Hoboken, N.J., USA.

17. Bonifacino, J. S., M. Dasso, J. Lippincott-Schwartz, J. B. Harford, K. M. Yamada, and (Editors). 2002. Current Protocols in Cell Biology. John Wiley & Son, Inc., Hoboken, N.J., USA.

18. Wilm, M., and M. Mann. 1996. Analytical properties of the nanoelectrospray ion source. Anal Chem. 68:1-8.

19. Engelborghs, S., and P. P. De Deyn. 2001. Biological and genetic markers of sporadic Alzheimer's disease. Acta Med Okayama. 55:55-63.

20. Papayannopoulos, I. A. 1995. The interpretation of collision-induced dissociation tandem mass spectra of peptides. Mass Spectrom Rev. 49-73.

21. Perkins, D. N., D. J. Pappin, D. M. Creasy, and J. S. Cottrell. 1999. Probability-based protein identification by searching sequence databases using mass spectrometry data. Electrophoresis. 20:3551-67.

1. A method for the detection of a neurological disease, preferably of Alzheimer's disease, or of a predisposition to such a disease by determining at least one DRES peptide corresponding to Seq. ID 1 to 44 and 47 to 57 or a mutant thereof which differs in a maximum of two amino acids from the corresponding unmutated DRES sequence or an amino acid sequence which is at least 70% homologous thereto, said peptides may be chemically or enzymatically modified, or post-translationally modified, preferably a phosphorylated, a sulfate, an oxidized, a C-terminally amidated peptide or a peptide having a pyroglutamate modification in an individual's liquid biological sample.

2. The method as claimed in claim 1, wherein the liquid biological sample a) is cerebrospinal fluid, serum, plasma, whole blood, urine, tear fluid, lymph, synovial fluid, sputum, stool or a homogenized cell sample or homogenized tissue sample, and/or b) is fractionated by chromatography before the identification, preferably by reverse phase chromatography, and/or c) is fractionated before the identification by precipitation reactions or extraction methods.

3. The method as claimed in claim 1, which a) is carried out in combination with other diagnostic methods to increase the sensitivity and/or specificity thereof, or b) is used for the identification of subgroups of individuals with a neurological disease, preferably with Alzheimer's disease, who respond to particular types of therapy (stratification).

4. The method according to claim 1, further comprising the detection of at least one peptide according to Seq. ID 58 to 85 or a peptide mutant which differs in a maximum of two amino acids from the corresponding unmutated sequence or an amino acid sequence which is at least 70% homologous thereto, said peptides may be chemically or enzymatically modified, or post-translationally modified, preferably a phosphorylated, a sulfated, an oxidized, a C-terminally amidated peptide or a peptide having a pyroglutamate modification.

5. The method according to claim 1, wherein at least one peptide of each of DRES peptides (Seq. ID 1 to 44 or 47 to 57), secretogranin 5 peptides (Seq. ID 58 to 60), Secretogranin 2 peptides (Seq. ID 61 to 71), Chromogranin A peptides (Seq. ID 72 to 85) is detected.

6. The method according to claim 5, wherein the peptides according to Seq. tD 1, 58, 61 and 85 are detected.

7. A method for the detection of a neurological disease, preferably of Alzheimer's disease or of a predisposition to such a disease by determining at least one peptide derived from the amino acid sequence corresponding to GeneBank Accession No. NM 001819 (Seq-ID. 45) and at least one peptide derived from any one of the sequences corresponding to GeneBank Accession Nos. A28468, Chromogranin A (Seq-ID. 88), NP003460, secretogranin 2 (Seq-ID. 86), or NP003011, secretogranin 5 (Seq-ID. 87) or an amino acid sequence which is at least 70% homologous thereto, said peptides may be chemically or enzymatically modified, or post-translationally modified, preferably a phosphorylated, a sulfate, an oxidized, a C-terminally amidated peptide or a peptide having a pyroglutamate modification in an individual's liquid biological sample.

8. A method according to claim 4, wherein at least three, preferably four different peptides derived from at least three, preferably four different proteins of said proteins mentioned therein are detected.

9. The method as claimed in claim 1, wherein the determination is conducted by applying an activity assay, an immunological, a molecular biological, a physical or a chemical assay.

10. The method as claimed in claim 9, wherein the mass of the peptides is used for physical determination of the peptides, preferably with use of mass spectrometry.

11. The method as claimed in claim 10, wherein the determination of the masses encompasses at least one of the theoretical monoisotopic masses from 4605. 0/4620. 1/4392.9/4107. 8/4321. 9/2853. 3/2368. 1/4619. 0/4335. 9/3246.5/#686.3/#934. 4 891. 4/6433. 7/4583. 1/4427. 0/2522. 1/>835. 4 805. 3 864. 3/3202. 4/#774.3/#933. 4/1985.8/#991. 4/1992. 8/&gt; 976. 4/4750. 2/#906.3/#892.5/6499.0/6264.9/5565.6/5067.3/4867.2 /4791. 2 835. 3 930. 4/2 1001. 4/6970.3/#862.4/1588.9/919. 4 942. 6 1288,6 1677, 8/ 1268,6/1422,7/1096, 5/1341,5/1023, 6/1233, 7/2991,4 2899, 3/4469/9653, 4/9724, 4/9723, 5/ 5730,6/5061, 5/2065,1/2490, 4 2003, 1/1890, 1/3905, 8/1219, 6 4152,9 2385,2 3100,5 1829,9 1508,7 4180 2030 2159,1 4796,4 4867,4 4657,3/3086, 6/1843,9/1508, 8/4162,9/2145, 0 2274, 0/ 4911, 4/4982, 5/4738, 3/#975, 5 &gt; 1111, 5 &gt; 952,4/3510, 7/1500, 6/2448, 3 or 3590,7 Dalton.

12. The method as claimed in claim 9, wherein an immunological assay, preferably an ELISA (enzyme-linked immunosorbent assay), a radioimmunoassay, a protein chip assay or a Western blot is employed.

13. A diagnostic kit comprising at least one compound for the detection of one of the compounds as defined in claim 1.

14. A test kit for carrying out a method according to claim 1, which comprises as a minimum a) an antibody fragment or an antibody which is directed against a peptide selected from the group consisting of SBO ID NOS: 1-44, 47-57, and 58-85, or against a peptide derived from the amino acid sequence represented by SEQ ID NOS: 45, or 86-88 and wherein the antibody or the antibody fragment is present in immobilized or labelled form, or in a form which makes immobilization or labelling possible and/or b) wherein said peptide is used as a standard or control.

15. A substance, which a) is a DRES peptide corresponding to Seq. ID 1 to ID 13, ID 15 to ID 20, ID 22 to ID 39, ID 41 to ID 44 or ID 47 to 57, or b) is a mutant of a), where the mutant preferably differs in a maximum of two amino acids from the corresponding unmutated sequence, or c) is a peptide which has at least 70% homology with the amino acid sequence of the DRES peptides corresponding to Seq. ID Nos. 1 to 44 or 47 to 57, or d) is a peptide corresponding to Seq. ID Nos. 58 to 85 or a peptide which has at least 70% homology with said peptides e) is a chemically or enzymatically modified, or post-translationally modified peptide corresponding to Seq. ID 1 to 44 and ID 47 to 57 or corresponding to b) to d), preferably a phosphorylated, a sulfate, an oxidized, a C-terminally amidated peptide or a peptide having a pyroglutamate or biotin modification, or f) is a fusion peptide or fusion protein which, besides the sequences according to a) to d), comprises further amino acid sequences, preferably sequences such as, for example, an HIV Tat or a His tag sequence, which make it possible for the fusion molecule to be more easily isolated, detected or transferred from the extracellular into the intracellular space, or g) is a peptidomimetic of one of the substances mentioned under a) to f), or h) is an antibody which binds at least one of the substances mentioned under a) to e), or i) is a salt of one of the substances mentioned under a) to g).

16. (canceled)

17. A preparation comprising at least one substance as defined in claim 15, further comprising a transport unit, preferably transport peptides such as, for example, HIV Tat, polymers preferably polyethylene glycol, enteric- coated capsules, liposomes, whereby ingredients of the preparation a) are able to cross the blood-brain barrier and/or the blood-CSF barrier, or b) are able to pass from the extracellular space into the intracellular space, or c) are optimised for specific administration routes, in particular for administration into the blood stream, the gastrointestinal tract, the urogenital tract, the lymphatic system, the subarachnoid space, for topical application, for inhalation or for direct injection into tissue such as, for example, muscle tissue, adipose tissue or brain for in vitro treatment of cells.

18. (canceled)

19. A medicinal product for therapy, diagnosis or prophylaxis comprising at least one substance according to claim 15, and at least one further pharmacologically acceptable substance, preferably a preservative, a bulking agent, a solvent, a color, a flavoring or a fragrance.

20. (canceled)

21. A screening method for identifying substances able to reduce or to enhance the concentration of at least one of the substances in claim 15, or
b) receptors which bind at least one of the substances in claim 15a) to e) and 15g), or
c) agonists or antagonists of at least one of the substances in claim 15, comprising the steps of
providing a substance of claim 15 or a nucleic acid encoding said substance and
bringing said substance or said nucleic acid encoding said substance into association with a test substance, and
determining whether said test substance has the ability to modulate the expression or activity of said substance.

22. A nucleic acid, wherein said nucleic acid is selected from the group consisting of
a) a first nucleic acid which codes for
i) a DRES peptide corresponding to Seq. ID 1 to ID 13, ID 15 to ID 20, ID 22 to ID 39, ID 41 to ID 44 or ID 47 to 57, or
ii) is a mutant of said DRES peptide, where the mutant preferably differs in a maximum of two amino acids from the corresponding unmutated sequence, or
iii) a peptide which has at least 70% homology with the amino acid sequence of the DRES peptides corresponding to Seq. ID Nos. 1 to 44 or 47 to 57, or
iv) a peptide corresponding to Seq. ID Nos. 58 to 85 or a peptide which has at least 70% homology with said peptides; or
b) a second nucleic acid which is complementary to said first nucleic acid;
c) a third nucleic acid, wherein said third nucleic acid is a ribozyme, an antisense nucleic acid, a triplex-forming nucleic acid, an RNAi nucleic acid or another nucleic acid which specifically binds to and inactivates said first or second nucleic acid;
d) a fourth nucleic acid which hybridizes specifically with any one of said first, second or third nucleic acids; and
e) a linear or circular vector which comprises at least one of said first, second third or fourth nucleic acids;
or a salt thereof.

23. The nucleic acid of claim 22, wherein said nucleic acid further comprises an element selected from the group consisting of promoters and sequences which convey antibiotic resistance.

24. A medicinal product for therapy, diagnosis or prophylaxis comprising at least one substance according to claim 22, and at least one further pharmacologically acceptable substance, preferably a preservative, a bulking agent, a solvent, a color, a flavoring or a fragrance.

25. The medicinal product of claim 19, wherein said substance further comprises a transport unit.


SEQUENCE LISTING (88 sequences)

SEQ ID NO: 1 - protein, 45 aa, Homo sapiens
DPADASEAHESSSRGEAGAPGEEDIQGPTKADTEKWAEGGGHSRE

SEQ ID NO: 2 - protein, 45 aa, Homo sapiens
ADASEAHESSSRGEAGAPGEEDIQGPTKADTEKWAEGGGHSRERA

SEQ ID NO: 3 - protein, 43 aa, Homo sapiens
ADASEAHESSSRGEAGAPGEEDIQGPTKADTEKWAEGGGHSRE

SEQ ID NO: 4 - protein, 41 aa, Homo sapiens
ADASEAHESSSRGEAGAPGEEDIQGPTKADTEKWAEGGGHS

SEQ ID NO: 5 - protein, 42 aa, Homo sapiens
DASEAHESSSRGEAGAPGEEDIQGPTKADTEKWAEGGGHSRE

SEQ ID NO: 6 - protein, 29 aa, Homo sapiens
ADASEAHESSSRGEAGAPGEEDIQGPTKA

SEQ ID NO: 7 - protein, 22 aa, Homo sapiens
DIQGPTKADTEKWAEGGGHSRE

SEQ ID NO: 8 - protein, 45 aa, Homo sapiens
DPADATEAHESSSRGEAGAPGEEDIQGPTKADTEKWAEGGGHSRE

SEQ ID NO: 9 - protein, 42 aa, Homo sapiens
DATEAHESSSRGEAGAPGEEDIQGPTKADTEKWAEGGGHSRE

SEQ ID NO: 10 - protein, 28 aa, Homo sapiens
ADEPQWSLYPSDSQVSEEVKTRHSEKSQ

SEQ ID NO: 11 - protein, 8 aa, Homo sapiens
  MISC_FEATURE: N-terminal extension r1 possible; r1 represents a sequence which corresponds to the sequence or parts of the sequence of SG1 from amino acid 99 to 88; C-terminal extension r2 possible, r2 represents the SG1 protein sequence from amino acid 108 to 134
SRGEAGAP

SEQ ID NO: 12 - protein, 8 aa, Homo sapiens
  MISC_FEATURE: N-terminal extension r3 possible; r3 represents a sequence which corresponds to the sequence or parts of the sequence of SG1 from amino acid 118 to 88; C-terminal extension r4 possible, r4 represents the SG1 protein sequence from amino acid 127 to 161
DTEKWAEG

SEQ ID NO: 13 - protein, 8 aa, Homo sapiens
  MISC_FEATURE: N-terminal extension r5 possible; r5 represents a sequence which corresponds to the sequence or parts of the sequence of SG1 from amino acid 144 to 134; C-terminal extension r6 possible, r6 represents the SG1 protein sequence from amino acid 153 to 161
DSQVSEEV

SEQ ID NO: 14 - protein, 59 aa, Homo sapiens
SETHAAGHSQEKTHSREKSSQESGEEAGSQENHPQESKGQPRSQEESEEGEEDATSEVD

SEQ ID NO: 15 - protein, 42 aa, Homo sapiens
SETHAAGHSQEKTHSREKSSQESGEEAGSQENHPQESKGQPR

SEQ ID NO: 16 - protein, 41 aa, Homo sapiens
SETHAAGHSQEKTHSREKSSQESGEEAGSQENHPQESKGQP

SEQ ID NO: 17 - protein, 23 aa, Homo sapiens
SKGQPRSQEESEEGEEDATSEVD

SEQ ID NO: 18 - protein, 8 aa, Homo sapiens
  MISC_FEATURE: N-terminal extension r7 possible; r7 represents a sequence which corresponds to the sequence or parts of the sequence of SG1 from amino acid 219 to 217; C-terminal extension r8 possible, r8 represents the SG1 protein sequence from amino acid 228 to 275
HAAGHSQE

SEQ ID NO: 19 - protein, 8 aa, Homo sapiens
  MISC_FEATURE: N-terminal extension r9 possible; r9 represents a sequence which corresponds to the sequence or parts of the sequence of SG1 from amino acid 239 to 217; C-terminal extension r10 possible, r10 represents the SG1 protein sequence from amino acid 248 to 275
GEEAGSQE

SEQ ID NO: 20 - protein, 8 aa, Homo sapiens
  MISC_FEATURE: N-terminal extension r11 possible; r11 represents a sequence which corresponds to the sequence or parts of the sequence of SG from amino acid 262 to 217; C-terminal extension r12 possible, r12 represents the SG1 protein sequence from amino acid 271 to 323
SEEGEEDA

SEQ ID NO: 21 - protein, 31 aa, Homo sapiens
SSQGGSLPSEEKGHPQEESEESNVSMASLGE

SEQ ID NO: 22 - protein, 8 aa, Homo sapiens
  MISC_FEATURE: N-terminal extension r13 possible; r13 represents a sequence which corresponds to the sequence or parts of the sequence of SG1 from amino acid 295 to 293; C-terminal extension r14 possible, r14 represents the SG1 protein sequence from amino acid 304 to 323
GGSLPSEE

SEQ ID NO: 23 - protein, 8 aa, Homo sapiens
  MISC_FEATURE: N-terminal extension r15 possible; r15 represents a sequence which corresponds to the sequence or parts of the sequence of SG1 from amino acid 306 to 293; C-terminal extension r16 possible, r16 represents the SG1 protein sequence from amino acid 315 to 323
PQEESEES

SEQ ID NO: 24 - protein, 16 aa, Homo sapiens
DHHSTHYRASEEEPEY

SEQ ID NO: 25 - protein, 8 aa, Homo sapiens
  MISC_FEATURE: N-terminal extension r17 possible; r17 represents a sequence which corresponds to the sequence or parts of the sequence of SG1 from amino acid 329 to 326; C-terminal extension r18 possible, r18 represents the SG1 protein sequence from amino acid 338 to 341
THYRASEE

SEQ ID NO: 26 - protein, 16 aa, Homo sapiens
YRAPRPQSEESWDEED

SEQ ID NO: 27 - protein, 8 aa, Homo sapiens
  MISC_FEATURE: N-terminal extension r19 possible; r19 represents a sequence which corresponds to the sequence or parts of the sequence of SG1 from amino acid 374 to 366; C-terminal extension r20 possible, r20 represents the SG1 protein sequence from amino acid 383 to 387
PQSEESWD

SEQ ID NO: 28 - protein, 42 aa, Homo sapiens
NYPSLELDKMAHGYGEESEEERGLEPGKGRHHRGRGGEPRAY

SEQ ID NO: 29 - protein, 8 aa, Homo sapiens
  MISC_FEATURE: N-terminal extension r21 possible; r21 represents a sequence which corresponds to the sequence or parts of the sequence of SG1 from amino acid 398 to 388; C-terminal extension r22 possible, r22 represents the SG1 protein sequence from amino acid 407 to 429
HGYGEESE

SEQ ID NO: 30 - protein, 8 aa, Homo sapiens
  MISC_FEATURE: N-terminal extension r23 possible; r23 represents a sequence which corresponds to the sequence or parts of the sequence of SG1 from amino acid 410 to 388; C-terminal extension r24 possible, r24 represents the SG1 protein sequence from amino acid 419 to 429
LEPGKGRH

SEQ ID NO: 31 - protein, 55 aa, Homo sapiens
HPQGAWKELDRNYLNYGEEGAPGKWQQQGDLQDTKENREEARFQDKQYSSHHTAE

SEQ ID NO: 32 - protein, 53 aa, Homo sapiens
QGAWKELDRNYLNYGEEGAPGKWQQQGDLQDTKENREEARFQDKQYSSHHTAE

SEQ ID NO: 33 - protein, 47 aa, Homo sapiens
LDRNYLNYGEEGAPGKWQQQGDLQDTKENREEARFQDKQYSSHHTAE

SEQ ID NO: 34 - protein, 43 aa, Homo sapiens
YLNYGEEGAPGKWQQQGDLQDTKENREEARFQDKQYSSHHTAE

SEQ ID NO: 35 - protein, 41 aa, Homo sapiens
YLNYGEEGAPGKWQQQGDLQDTKENREEARFQDKQYSSHHT

SEQ ID NO: 36 - protein, 41 aa, Homo sapiens
NYGEEGAPGKWQQQGDLQDTKENREEARFQDKQYSSHHTAE

SEQ ID NO: 37 - protein, 8 aa, Homo sapiens
  MISC_FEATURE: N-terminal extension r25 possible; r25 represents a sequence which corresponds to the sequence or parts of the sequence of SG1 from amino acid 472 to 459; C-terminal extension r26 possible, r26 represents the SG1 protein sequence from amino acid 481 to 513
NYGEEGAP

SEQ ID NO: 38 - protein, 8 aa, Homo sapiens
  MISC_FEATURE: N-terminal extension r27 possible; r27 represents a sequence which corresponds to the sequence or parts of the sequence of SG1 from amino acid 483 to 459; C-terminal extension r28 possible, r28 represents the SG1 protein sequence from amino acid 492 to 513
QQQGDLQD

SEQ ID NO: 39 - protein, 8 aa, Homo sapiens
  MISC_FEATURE: N-terminal extension r29 possible; r29 represents a sequence which corresponds to the sequence or parts of the sequence of SG1 from amino acid 459 to 500; C-terminal extension r30 possible, r30 represents the SG1 protein sequence from amino acid 509 to 513
FQDKQYSS

SEQ ID NO: 40 - protein, 60 aa, Homo sapiens
SAEFPDFYDSEEPVSTHQEAENEKDRADQTVLTEDEKKELENLAAMDLELQKIAEKFSQR

SEQ ID NO: 41 - protein, 8 aa, Homo sapiens
  MISC_FEATURE: N-terminal extension r31 possible; r31 represents a sequence which corresponds to the sequence or parts of the sequence of SG1 from amino acid 624 to 617; C-terminal extension r32 possible, r32 represents the SG1 protein sequence from amino acid 633 to 677
DSEEPVST

SEQ ID NO: 42 - protein, 13 aa, Homo sapiens
LELQKIAEKFSQR

SEQ ID NO: 43 - protein, 8 aa, Homo sapiens
  MISC_FEATURE: N-terminal extension r33 possible; r33 represents a sequence which corresponds to the sequence or parts of the sequence of SG1 from amino acid 643 to 617; C-terminal extension r34 possible, r34 represents the SG1 protein sequence from amino acid 652 to 677
DQTVLTED

SEQ ID NO: 44 - protein, 8 aa, Homo sapiens
  MISC_FEATURE: N-terminal extension r35 possible; r35 represents a sequence which corresponds to the sequence or parts of the sequence of SG1 from amino acid 663 to 617; C-terminal extension r36 possible, r36 represents the SG1 protein sequence from amino acid 672 to 677
LELQKIAE

SEQ ID NO: 45 - protein, 677 aa, Homo sapiens
MQPTLLLSLLGAVGLAAVNSMPVDNRNHNEGMVTRCIIEVLSNALSKSSAPPITPECRQVLKTSRKDVKDKETTENENTKFEVRLLRDPADASEAHESSSRGEAGAPGEEDIQGPTKADTEKWAEGGGHSRERADEPQWSLYPSDSQVSEEVKTRHSEKSQREDEEEEEGENYQKGERGEDSSEEKHLEEPGETQNAFLNERKQASAIKKEELVARSETHAAGHSQEKTHSREKSSQESGEEAGSQENHPQESKGQPRSQEESEEGEEDATSEVDKRRTRPRHHHGRSRPDRSSQGGSLPSEEKGHPQEESEESNVSMASLGEKRDHHSTHYRASEEEPEYGEEIKGYPGVQAPEDLEWERYRGRGSEEYRAPRPQSEESWDEEDKRNYPSLELDKMAHGYGEESEEERGLEPGKGRHHRGRGGEPRAYFMSDTREEKRFLGEGHHRVQENQMDKARRHPQGAWKELDRNYLNYGEEGAPGKWQQQGDLQDTKENREEARFQDKQYSSHHTAEKRKRLGELFNPYYDPLQWKSSHFERRDNMNDNFLEGEEENELTLNEKNFFPEYNYDWWEKKPFSEDVNWGYEKRNLARVPKLDLKRQYDRVAQLDQLLHYRKKSAEFPDFYDSEEPVSTHQEAENEKDRADQTVLTEDEKKELENLAAMDLELQKIAEKFSQRG

SEQ ID NO: 46 - DNA, 2454 bp, Homo sapiens
ccaggaggcacgctggttttccggggccgctccatcgcgccttcctcctgcgcctcgcttctccggtccagccgccatcttcctttccgcacaggggccgccgagcggggccatgcagccaacgctgcttctcagcctcctgggagccgtggggctggcggctgtcaattccatgccagtggataacaggaaccacaatgaaggaatggtgactcgctgcatcattgaggtcctctcaaatgccttgtcgaagtccagcgctccacccatcacccctgagtgccgccaagtcctgaagacgagtagaaaagacgtcaaagacaaagagacaactgaaaatgaaaacacaaagtttgaagtaagattgttaagagacccagctgatgcctcggaagcccacgagtcctccagcaggggagaggcaggagccccaggggaggaggacatccaaggcccaacaaaggcagacacagagaaatgggcagagggaggcgggcacagccgagagcgagcggatgagccccagtggagcctctatccctccgacagccaagtctctgaagaagtgaagacacgccattctgagaagagccagagagaggatgaggaggaggaggagggagagaactatcaaaaaggggagcgaggggaagatagcagtgaagagaaacaccttgaagagccaggagagacacaaaacgcttttctcaatgaaagaaagcaggcttcagctataaaaaaagaggagttagtggccagatcggaaacacatgctgccgggcattctcaggagaagacacatagccgagagaagagtagccaggagagtggagaggaggcagggagccaggagaatcacccccaggagtctaaaggccaaccccgaagccaggaagaatctgaggaaggtgaggaagatgccacctctgaggtggacaaacgacgcacgaggcccagacaccaccacgggaggagcaggcccgacaggtcctctcaaggagggagtcttccctctgaggaaaagggacacccccaggaggaatctgaggagtcaaacgtcagcatggccagtttaggggaaaagagggaccaccattcaacccactacagggcttcagaggaagaacctgaatatggagaagaaataaagggttatccaggcgtccaggcccctgaggacctggagtgggagcgctataggggcagaggaagtgaagaatacagggctccaagacctcagagtgaggagagttgggatgaggaggacaagagaaactaccccagcttagagcttgataagatggcacatggatatggtgaagaaagtgaggaagagaggggccttgagccgggaaagggacgccatcacagaggcaggggaggggagccacgtgcctatttcatgtctgacaccagagaagagaaaaggttcttgggtgaaggacaccaccgtgtccaagaaaaccagatggacaaggcaaggaggcatccacaaggtgcgtggaaagagctggacagaaattatctcaactacggtgaggaaggagccccagggaagtggcagcagcagggagacctgcaggacactaaagaaaacagggaggaagctaggtttcaagataaacaatatagctcccatcacacagctgaaaagaggaagagattaggggaactgttcaacccatactacgaccctctccagtggaagagcagccattttgaaagaagagacaacatgaatgacaattttctcgagggtgaggaggaaaatgagctgaccttgaacgagaagaatttcttcccagaatacaactatgactggtgggagaaaaagcccttctctgaggatgtgaactgggggtatgagaagagaaacctcgccagggtccccaagctggacctgaaaaggcaatatgacagggtggcccaactggaccagctccttcactacaggaagaagtcagctgagtttccagacttctatgattctgaggagccggtgagcacccaccaggaggcagaaaatgaaaaggacagggctgaccagacagtcctgacagaggacgagaaaaaagaactcgaaaacttggctgcaatggatttggaactacagaagatagctgagaaattcagccaaaggggctgactgtcattggagcggtgggcactgttaagaagcagccatcacatgatctgtttttcaccacttcactgaaagacaccatttatatacccaagggcagaaagtagaacttactattcattaaatgtttgacacaattggaattgtctttaatttctgtcagaatgctattgaaaatgtgaattgcatgacttgtagcatattcttttctgcaaaatagacatattaacatgcttatgacaatgactgtgctactgtctttggaaaaatgtttgtctcagttggaaataataaaagattcacctgagacc

SEQ ID NO: 47 - protein, 11 aa, Homo sapiens
GSEEYRAPRPQ

SEQ ID NO: 48 - protein, 14 aa, Homo sapiens
EGHHRVQENQMDKA

SEQ ID NO: 49 - protein, 8 aa, Homo sapiens
  MISC_FEATURE: N-terminal extension r37 possible; r37 represents a sequence which corresponds to the sequence or parts of the sequence of SG1 from amino acid 443 to 440; C-terminal extension r38 possible, r38 represents the SG1 protein sequence from amino acid 452-456
GHHRVQEN

SEQ ID NO: 50 - protein, 10 aa, Homo sapiens
LFNPYYDPLQ

SEQ ID NO: 51 - protein, 11 aa, Homo sapiens
YYDPLQWKSSH

SEQ ID NO: 52 - protein, 9 aa, Homo sapiens
DPLQWKSSH

SEQ ID NO: 53 - protein, 8 aa, Homo sapiens
  MISC_FEATURE: N-terminal extension r39 possible; r39 represents a sequence which corresponds to the sequence or parts of the sequence of SG1 from amino acid 524 to 518; C-terminal extension r40 possible, r40 represents the SG1 protein sequence from amino acid 533-537
YYDPLQWK

SEQ ID NO: 54 - protein, 11 aa, Homo sapiens
PFSEDVNWGYE

SEQ ID NO: 55 - protein, 8 aa, Homo sapiens
  MISC_FEATURE: N-terminal extension r41 possible; r41 represents a sequence which corresponds to the sequence or parts of the sequence of SG1 from amino acid 575 to 575; C-terminal extension r42 possible, r42 represents the SG1 protein sequence from amino acid 584-585
FSEDVNWG

SEQ ID NO: 56 - protein, 9 aa, Homo sapiens
LARVPKLDL

SEQ ID NO: 57 - protein, 10 aa, Homo sapiens
QKIAEKFSQR

SEQ ID NO: 58 - protein, 22 aa, Homo sapiens
SVNPYLQGQRLDNVVAKKSVPH

SEQ ID NO: 59 - protein, 31 aa, Homo sapiens
SVNPYLQGQRLNDVVAKKSVPHFSDEDKDPE

SEQ ID NO: 60 - protein, 13 aa, Homo sapiens
SVPHFSDEDKDPE

SEQ ID NO: 61 - protein, 38 aa, Homo sapiens
GQGSSEDDLQEEEQIEQAIKEHLNQGSSQETDKLAPVS

SEQ ID NO: 62 - protein, 20 aa, Homo sapiens
MKVLEYLNQEKAEKGREHIA

SEQ ID NO: 63 - protein, 28 aa, Homo sapiens
SGQLGIQEEDLRKESKDQLSDDVSKVIA

SEQ ID NO: 64 - protein, 16 aa, Homo sapiens
FPVGPPKNDDTPNRQY

SEQ ID NO: 65 - protein, 13 aa, Homo sapiens
NQEKAEKGREHIA

SEQ ID NO: 66 - protein, 37 aa, Homo sapiens
VPGQGSSEDDLQEEEQIEQAIKEHLNQGSSQETDKLP

SEQ ID NO: 67 - protein, 18 aa, Homo sapiens
FPVGPPKNDDTPNRQYWD

SEQ ID NO: 68 - protein, 19 aa, Homo sapiens
FPVGPPKNDDTPNRQYWDE

SEQ ID NO: 69 - protein, 41 aa, Homo sapiens
FPVGPPKNDDTPNRQYWDEDLLMKVLEYLNQEKAEKGREHI

SEQ ID NO: 70 - protein, 42 aa, Homo sapiens
FPVGPPKNDDTPNRQYWDEDLLMKVLEYLNQEKAEKGREHIA

SEQ ID NO: 71 - protein, 40 aa, Homo sapiens
VGPPKNDDTPNRQYWDEDLLMKVLEYLNQEKAEKGREHIA

SEQ ID NO: 72 - protein, 27 aa, Homo sapiens
SEVLENQSSQAELKEAVEEPSSKDVME

SEQ ID NO: 73 - protein, 27 aa, Homo sapiens
EDSKEAEKSGEATDGARPQALPEPMQE

SEQ ID NO: 74 - protein, 43 aa, Homo sapiens
EDSKEAEKSGEATDGARPQALPEPMQESKAEGNNQAPGEEEEE

SEQ ID NO: 75 - protein, 91 aa, Homo sapiens
EDSKEAEKSGEATDGARPQALPEPMQESKAEGNNQAPGEEEEEEEEATNTHPPASLPSQKYPGPQAEGDSEGLSQGLVDREKGLSAEPGWQ

SEQ ID NO: 76 - protein, 92 aa, Homo sapiens
EDSKEAEKSGEATDGARPQALPEPMQESKAEGNNQAPGEEEEEEEEATNTHPPASLPSQKYPGPQAEGDSEGLSQGLVDREKGLSAEPGWQA

SEQ ID NO: 77 - protein, 92 aa, Homo sapiens
DSKEAEKSGEATDGARPQALPEPMQESKAEGNNQAPGEEEEEEEEATNTHPPASLPSQKYPGPQAEGDSEGLSQGLVDREKGLSAEPGWQAK

SEQ ID NO: 78 - protein, 54 aa, Homo sapiens
SKEAEKSGEATDGARPQALPEPMQESKAEGNNQAPGEEEEEEEEATNTHPPASL

SEQ ID NO: 79 - protein, 30 aa, Homo sapiens
AVVPQGLFRGGKSGELEQEEERLSKEWEDS

SEQ ID NO: 80 - protein, 44 aa, Homo sapiens
GYPEEKKEEEGSANRRPEDQELESLSAIEAELEKVAHQLQALRR

SEQ ID NO: 81 - protein, 19 aa, Homo sapiens
ESLSAIEAELEKVAHQLQA

SEQ ID NO: 82 - protein, 22 aa, Homo sapiens
ESLSAIEAELEKVAHQLQALRR

SEQ ID NO: 83 - protein, 17 aa, Homo sapiens
IEAELEKVAHQLQALRR

SEQ ID NO: 84 - protein, 16 aa, Homo sapiens
EAELEKVAHQLQALRR

SEQ ID NO: 85 - protein, 35 aa, Homo sapiens
HSGFEDELSEVLENQSSQAELKEAVEEPSSKDVME

SEQ ID NO: 86 - protein, 617 aa, Homo sapiens
MAEAKTHWLGAALSLIPLIFLISGAEAASFQRNQLLQKEPDLRLENVQKFPSPEMIRALEYIENLRQQAHKEESSPDYNPYQGVSVPLQQKENGDESHLPERDSLSEEDWMRIILEALRQAENEPQSAPKENKPYALNSEKNFPMDMSDDYETQQWPERKLKHMQFPPMYEENSRDNPFKRTNEIVEEQYTPQSLATLESVFQELGKLTGPNNQKRERMDEEQKLYTDDEDDIYKANNIAYEDVVGGEDWNPVEEKIESQTQEEVRDSKENIGKNEQINDEMKRSGQLGIQEEDLRKESKDQLSDDVSKVIAYLKRLVNAAGSGRLQNGQNGERATRLFEKPLDSQSIYQLIEISRNLQIPPEDLIEMLKTGEKPNGSVEPERELDLPVDLDDISEADLDHPDLFQNRMLSKSGYPKTPGRAGTEALPDGLSVEDILNLLGMESAANQKTSYFPNPYNQEKVLPRLPYGAGRSRSNQLPKAAWIPHVENRQMAYENLNDKDQELGEYLARMLVKYPEIINSNQVKRVPGQGSSEDDLQEEEQIEQAIKEHLNQGSSQETDKLAPVSKRFPVGPPKNDDTPNRQYWDEDLLMKVLEYLNQEKAEKGREHIAKRAMENM

SEQ ID NO: 87 - protein, 211 aa, Homo sapiens
MVSRMVSTMLSGLLFWLASGWTPAFAYSPRTPDRVSEADIQRLLHGVMEQLGIARPRVEYPAHQAMNLVGPQSIEGGAHEGLQHLGPFGNIPNIVAELTGDNIPKDFSEDQGYPDPPNPCPVGKTDDGCLENTPDTAEFSREFQLHQHLFDPEHDYPGLGKWNKKLLYEKMKGGERRKRRSVNPYLQGQRLDNVVAKKSVPHFSDEDKDPE

SEQ ID NO: 88 - protein, 457 aa, Homo sapiens [sequence omitted]